(19) United States
(12) Patent Application Publication
(10) Pub. No.: US 2009/0288227 A1
(43) Pub. Date: Nov. 19, 2009

(54) Improvements in or Relating to Starch Storage in Plants

(75) Inventors: Keith Lindsey, Durham (GB); Stuart Casson, Durham (GB)

Correspondence Address: MORGAN LEWIS & BOCKIUS LLP, 1111 PENNSYLVANIA AVENUE NW, WASHINGTON, DC, 20004, US

(73) Assignee: University of Durham, Durham (GB)

(21) Appl. No.: 12/301,167

(22) PCT Filed: May 18, 2007
(86) PCT No.: PCT/GB07/01833

§ 371 Date: Nov. 17, 2008

(30) Foreign Application Priority Data
May 18, 2006 (GB) 0609818.0
Jun. 24, 2006 (GB) 0612585.0

(51) Int. Cl. A01H 1/00; C07H 21/04; C12N 15/63; C12P 19/34; A01H 5/00

(52) U.S. Cl. 800/290; 536/24.1; 435/320.1; 435/91.1; 800/298

(57) ABSTRACT

The present invention provides an isolated fragment of the LEC1 promoter comprising a deletion, relative to the wild type LEC1 promoter, which isolated fragment possesses promoter activity in non-embryonic vegetative plant tissues in Arabidopsis, wherein the isolated fragment comprises at least 500 bases of the sequence shown in FIG. 1, or a functional equivalent thereof.

### FIELD OF THE INVENTION

This invention relates to starch storage in plants. More especially, the invention is concerned with polynucleotides, which can cause the increased expression of a gene involved in seed development resulting in, for example, increased production of storage compounds such as starch and triacylglycerols.

### BACKGROUND OF THE INVENTION

Starch is a major industrial product that has particular importance in the food industry. Plants represent a major source of starch, as it accumulates to high levels in storage organs such as seeds and tubers. In oil seed crops and their relatives, such as the Brassica family and the genetic model Arabidopsis, the accumulation of starch is predominantly restricted to the developing seed, in the embryo or ectoderm. In contrast, vegetative tissues (for example, leaves, hypocotyls and roots) do not accumulate significant levels of starch. Therefore, in such plants starch production, alongside the synthesis and accumulation of triacylglycerols (storage lipids) and storage proteins, can be considered a feature of embryonic development. Following germination of the embryo, these storage products are mobilised as an energy supply and used to support the growth of the young seedling.

The biochemistry and enzymology of starch biosynthesis has been well characterised. In essence, sugars produced during photosynthesis are polymerised to form insoluble starch granules that are stored in membrane-bound organelles. However, it has not yet been shown how the synthesis and accumulation of starch is activated in the embryo and suppressed in vegetative organs and tissues post-embryonically.

The early stages of embryogenesis in flowering plants involve the establishment of polarity, radial symmetry and cellular differentiation. In addition, the formation of shoot and root meristems determine the post-embryonic development of the plant (Laux et al, 2004 Plant Cell 16, S190-S202). During the later stages of embryogenesis, the nutrient stores required during germination are established. In addition, the process of desiccation occurs which prepares the embryo for dormancy (Raz et al, 2001 Development 128, 243-252). The transition between the early and later stages of embryogenesis is an important stage in the plant life cycle and is under the control of several key genes and plant growth regulators (Parcy et al, 1997 Plant Cell 9, 1265-1277; Ogas et al, 1997 Science 277, 91-94; Lotan et al, 1998 Cell 93, 1195-1205; Luerben et al, 1998 Plant J. 15, 755-764; Ogas et al, 1999 Proc. Natl. Acad. Sci. USA 96, 13839-13844; Raz et al, 2001 Development 128, 243-252; Stone et al, 2001 Proc. Natl. Acad. Sci: USA 98, 11806-11811).

The LEAFY COTYLEDON class of genes (LEC1, LEC2, FUSCA3, FUS3) have been identified as key regulators during the late stages of embryogenesis (Parcy et al, 1997 Plant Cell 9, 1265-1277; Lotan et al, 1998 Cell 93, 1195-1205; Leurben et al, 1998 Plant J. 15, 755-764; Stone et al, 2001 Proc. Natl. Acad. Sci. USA 98, 11806-11811). In particular, LEC1 encodes a transcription factor subunit which is related to the HAP3 subunit of the CCAAT binding factor family (Lotan et al, 1998 Cell 93, 1195-1205), whilst FUS3 and LEC2 encode B3 domain transcription factors (Luerben et al, 1998 Plant J. 15, 755-764; Stone et al, 2001 Proc. Natl. Acad. Sci. USA 98, 11806-11811). Loss-of-function mutations in each of these genes result in the production of embryos that are desiccation-intolerant and defective in the production of storage compounds. The mutant embryos also initiate post-germination processes, including premature activation of the shoot apical meristem. This has led to the suggestion that these genes play a role in inhibiting premature germination (Meinke et al, 1994 Plant Cell 6, 1049-1064). The cotyledons of the mutants demonstrate leaf-like features (such as the formation of trichomes), suggesting that these genes also function in the determination of organ identity.

In addition to functioning as regulators in the late stages of embryogenesis, the LEC genes play a role in regulating aspects of early embryogenesis. The suspensors of lec mutants (which act as a conduit between the embryo and maternal tissues) have been shown to develop abnormally. In the case of lec1-2 fus 3-3 double mutants, the suspensors can continue to proliferate and form secondary embryos, thus suggesting that LEC genes may act to maintain suspensor cell fate and inhibit the embryonic potential of the suspensors.

The expression of the LEC1 gene is limited to embryogenesis, whilst LEC2 and FUS3 genes are also expressed at low levels post-germination. Ectopic expression of LEC1 or LEC2 under the control of the CaMV35S promoter is sufficient to induce embryonic characteristics in vegetative tissue, suggesting that these genes are involved in the regulation of embryonic competence (Lotan et al, 1998 Cell 93, 1195-1205; Luerben et al, 1998 Plant J. 15, 755-764; Stone et al, 2001 Proc. Natl. Acad. Sci. USA 95, 11806-11811).

Previous analysis of lec1 mutants has shown that the LEC1 gene is required to specify embryonic organ identity (for example, lec1 mutants develop cotyledons with leaf-like features). In addition, the LEC1 gene is also involved in activating pathways that are involved in the accumulation of storage products (Meinke et al, 1994 Plant Cell 6, 1049-1064; West et al, 1994 Plant Cell 6, 1731-1745). Overexpression of LEC1 under the control of the CaMV35S promoter has been shown to result in a high degree of seedling lethality, wherein the seedlings demonstrate an embryo-like morphology (Lotan et al, 1998 Cell 93, 1195-1205). Those seedlings that survive produce embryo-like structures from vegetative tissues, indicating that expression of LEC1 is sufficient to induce embryonic developmental pathways in vegetative tissue.

Further evidence that the LEC genes are regulators of embryo development has been provided by studies of the PICKLE (PKL) gene that encodes a CHD3 chromatin-remodelling factor (Ogas et al, 1999 Proc. Natl. Acad. Sci. USA 96, 13839-13844). Mutations in the PKL gene result in the expression of embryonic traits in the vegetative root meristem (Ogas et al, 1997 Science 277, 91-94). Analysis of gene expression in pkl mutants reveals that they have high levels of LEC gene expression in vegetative tissue. The PKL gene is required for the repression of LEC genes during and after germination, thus preventing the activation of embryonic developmental pathways post-germination (Ogas et al, 1999 Proc. Natl. Acad. Sci. USA 96, 13839-13844; Dean Rider et al, 2003 Plant J. 35, 33-43). Interestingly, the pkl mutant phenotype shows low penetrance which can be influenced by growth regulators. In particular, the pkl phenotype is suppressed by gibberellins, whilst penetrance is increased by growth in the presence of the gibberellic acid biosynthetic inhibitor, uniconazole-P (Ogas et al, 1997 Science 277, 91-94). This, together with the fact that adult pkl plants display shoot phenotypes that are similar to gibberellic acid-deficient mutants, suggests that PKL is part of a gibberellic acid signalling pathway that promotes the transition from embryonic to vegetative development.

The involvement of growth regulators, particularly auxin, in both zygotic and somatic embryogenesis has been widely reported (Toonen and de Vries, Embryogenesis the generation of a plant (ed. T L Wang and A Cuming), 1996 Bios. Scientific Publishers, Oxford, pp. 173-189; Fischer-Iglesias et al, 2001 Plant J. 26, 115-129; Basu et al, 2002 Plant Physiol. 130, 292-302; Ribnicky et al, 2002 Planta 214, 505-509; Friml et al, 2003 Nature 426, 147-153). Synthetic auxin has been used in many species to induce somatic embryogenesis (Toonen and de Vries, 1996 Embryogenesis the generation of a plant (ed. T L Wang and A Cuming, 1996 Bios. Scientific Publishers, Oxford, pp 173-189), although the mechanism of auxin regulation is not well understood. In zygotic embryogenesis, the localisation and activities of auxin efflux carriers suggests that auxin distribution plays a crucial role in establishing the axes of polarity (Friml et al, 2003 Nature 426, 147-153). In particular, auxin is required for the polar expression of genes such as POLARIS (Topping and Lindsey, 1997 Plant Cell 9, 1713-1725; Casson et al, 2002 Plant Cell 14, 1705-1721). However, at present the relationship between auxin and the function of LEC is unknown.

### SUMMARY OF THE INVENTION

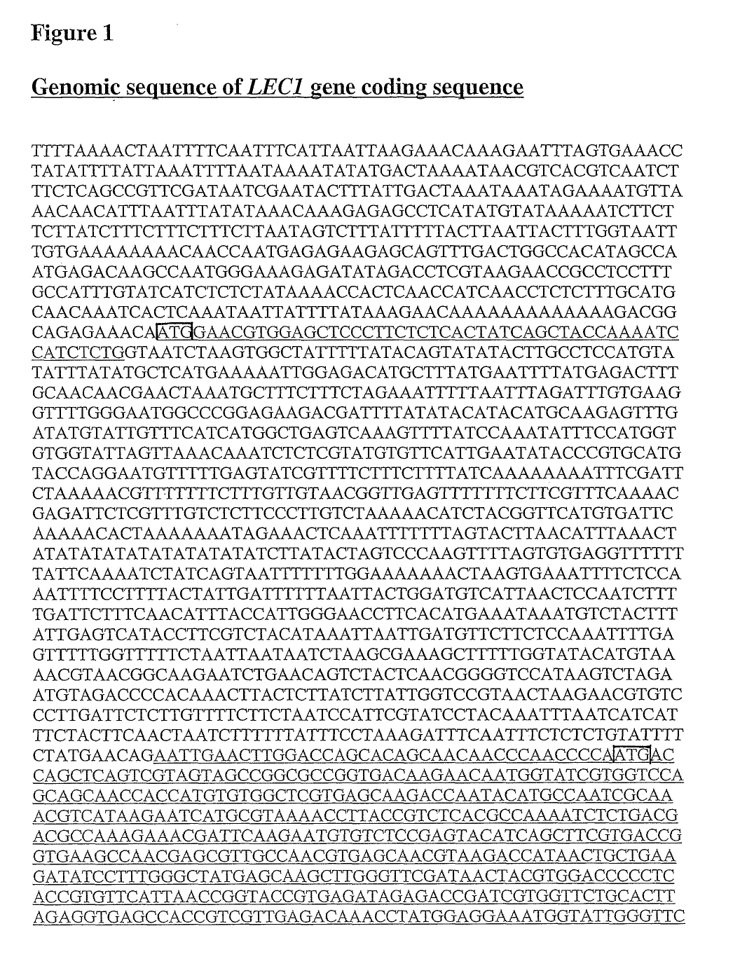

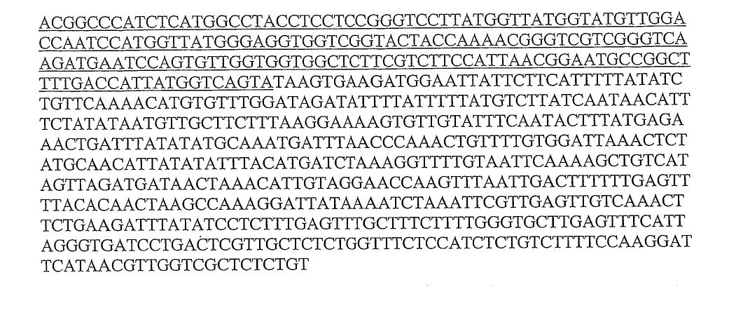

According to a first aspect, the present invention provides an isolated fragment of the LEC1 promoter comprising a deletion, relative to the wild type LEC1 promoter, which isolated fragment possesses promoter activity in non-embryonic vegetative plant tissues in Arabidopsis, wherein the isolated fragment comprises at least 500 bases of the sequence shown in FIG. 1, or a functional equivalent thereof, which term is defined below.

The isolated fragment of the present invention will comprise a sequence of at least 500 bases according to the sequence as shown in FIG. 1 or a functional equivalent thereof, which functional equivalent also possesses promoter activity in non-embryonic vegetative plant tissues of Arabidopsis and which exhibits 95% sequence identity over a portion of at least 500 bases of sequence as shown in FIG. 1 as determined by the sequence alignment program CLUSTAL W (Chenna et al, 2003, Nucleic Acids Res 31, 3497-3500).

The LEAFY COTYLEDON 1 (LEC1) gene is an important regulator required for the normal development of plants during the early and late stages of embryogenesis. In wild type plant cells, expression of LEC1 is restricted to embryogenesis and is partially repressed by the PICKLE (PKL) gene following germination in vegetative tissue. LEC1 is sufficient to induce embryonic development in vegetative cells and the repression of LEC1 expression is a key feature of the transition from embryonic to vegetative growth.

The wild type LEC1 promoter region comprises 1992 DNA base pairs 5′ of the LEC1 start codon and the terminator region comprises 770 DNA base pairs 3′ of the LEC1 stop codon (Kwong et al, 2003, The Plant Cell 15, 5-18).

The term “isolated” as used herein refers to a nucleic acid or polypeptide component which is substantially free from other components that normally interact with the polypeptide or nucleic acid as found in its natural environment or, if the polypeptide or nucleic acid is in its natural environment, the component has been altered by human intervention to form a composition and/or, in the case of a nucleic acid, has been placed at a locus in the cell other than the native locus.

As used herein, the term “fragment” refers to an incomplete portion of a nucleotide or amino acid sequence.

The term “promoter” includes reference to a region of DNA upstream from the transcription start site of a gene and “promoter activity” refers to the recognition and binding of RNA polymerase and other proteins to initiate transcription. Methods for detecting or measuring promoter activity (especially in Arabidopsis) may involve detecting or measuring the level of expression of a reporter gene, such as, for example, GFP. The isolated fragment of the present invention may, when introduced into a plant cell (typically an Arabidopsis cell) in non-embryonic tissue, in a suitable construct, lead to an increase in expression of an operably linked coding sequence relative to an otherwise identical plant cell comprising an equivalent construct comprising the full length wild type LEC1 promoter operably linked to the coding sequence. Thus, the promoter fragment of the invention may be considered to have promoter activity if it is able to induce significant expression of the operably linked coding sequence in non-embryonic tissues, whereas the complete, wild type LEC1 promoter is repressed in non-embryonic tissues. In preferred embodiments, the isolated fragment has promoter activity if it causes, in non-embryonic tissue, at least 10% of the level of expression caused by the same construct in embryos. Preferably, the isolated fragment causes, in non-embryonic tissue, at least 50% of the level of expression caused in embryos by the same construct, and more preferably about 100% of the level of expression caused in embryos.

For present purposes, the term “embryonic tissue” means tissue present in a seed from Arabidopsis up to 48 hours post germination. The term “non-embryonic” tissue means tissue from an Arabidopsis seedling at a time of thirty days post germination or longer.

The term “operable linkage” means, for the purposes of the present specification, that the promoter or promoter fragment is operably associated with a polynucleotide such that in suitable conditions the promoter or promoter fragment causes transcription of the associated polynucleotide.

There is deleted from the promoter fragment of the invention, relative to the complete wild type LEC1 promoter, typically at least 1000 bases, more preferably at least 1500 bases, and most preferably at least 2000 bases. FIG. 1 provides the base sequence of the complete LEC1 promoter. Thus, a promoter fragment in accordance with the present invention has a shorter sequence than that shown in FIG. 1. Typically, the promoter fragment of the invention comprises at least 1000, 1500 or 2000 bases fewer than the sequence shown in FIG. 1. In particular, there may be deleted from the promoter fragment of the invention, relative to the complete wild type LEC1 promoter sequence illustrated in FIG. 1, up to 2500, 3000, 3200 or even up to 3256 bases.

Typically, that portion of the wild type LEC1 promoter which is deleted from (and therefore absent from) the promoter fragment of the invention comprises that portion of the sequence located at position −1500 to −2000 of the full length promoter, wherein position −1 is the first base upstream from the start codon (i.e. the ATG codon). More especially, that portion of the wild type promoter which is deleted from the promoter fragment of the invention comprises that portion of the sequence located at position −1000 to −2500 of the full length promoter, preferably that portion of the sequence located at position −500 to −3500 of the full length promoter, wherein position −1 is the first base upstream from the start codon. In one particular embodiment, that portion of the wild type promoter which is deleted from the promoter fragment of the invention comprises the portion of the sequence located at position −436 to −3792 of the full length promoter, wherein position −1 is the first base upstream from the start codon. The start codon may be either of the ATG codons shown boxed in FIG. 1.

Preferably, the isolated fragment of the present invention is operably linked to the structural coding sequence of the LEC1 gene (AGI code At1g1970) or an effective portion thereof, such that when present in a non-embryonic plant cell, the expression of the LEC1 gene is increased and leads to an increase in lec transcript abundance in e.g. seedlings and other post-embryonic stages of development. Equally, the promoter fragment of the invention could be operably linked to any coding sequence, the expression of which is desired to be regulated by the promoter fragment of the invention, especially a plant gene coding sequence.

The Arabidopsis Genome Initiative (AGI) is an international collaboration to sequence the genome of the model plant Arabidopsis thaliana. Gene sequences obtained from A. thaliana are given a specific AGI code.

Other genes that may advantageously be expressed using the LEC1 promoter include embryonic identity genes such as LEC2 (AGI code At1g28300), FUS 3 (AGI code At3g26790), AB13 (AGI code At3g24650) and At5g17430, and their homologues from other plants.

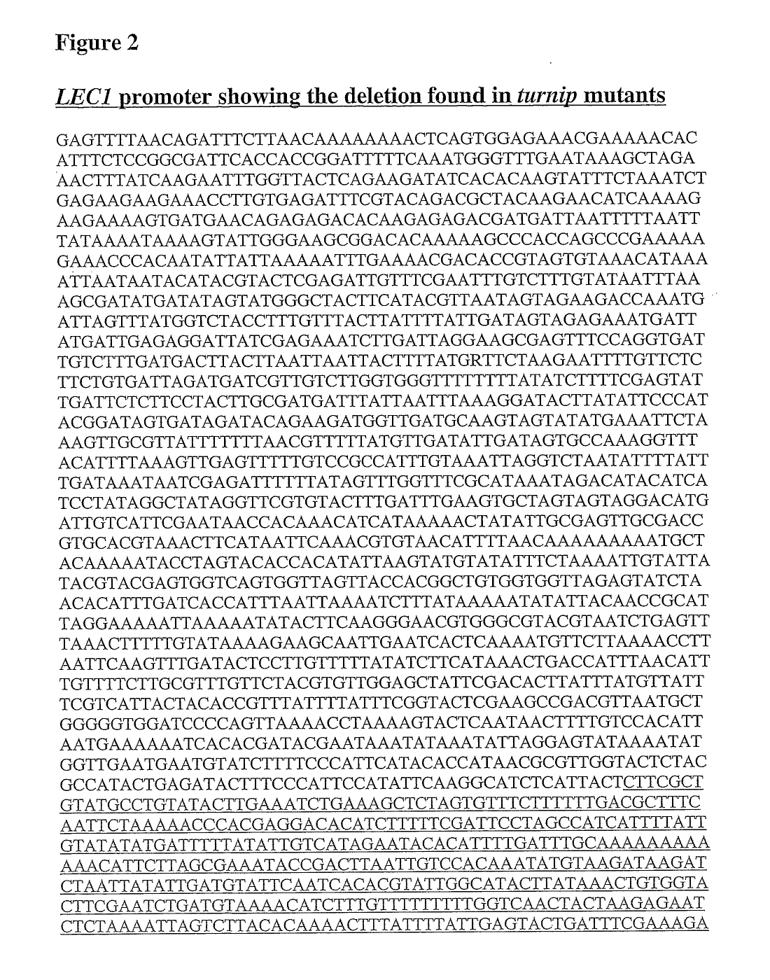

Preferably, the LEC1 polypeptide expressed following operable linkage of the promoter fragment of the present invention and the LEC1 coding sequence has at least 80% sequence identity to the wild type LEC1 coding sequence shown in FIG. 2. More preferably, the LEC1 polypeptide in accordance with the invention has at least 90% sequence identity and most preferably at least 95% sequence identity compared to the wild type LEC1 polypeptide coding sequence shown in FIG. 2.

In one embodiment, overexpression of the LEC1 gene in plant cells under the influence of the promoter fragment of the first aspect of the invention causes the accumulation of starch and/or oil or fatty acids and the like in vegetative tissues.

It is known that LEC1 expression is repressed in non-embryonic tissues (Ogas et al., 1999 Proc. Natl. Acad. Sci. U.S.A. 96, 13839-13844; Dean Rider et al, 2003 Plant J. 35, 33-43). The present inventors have found that the incomplete portion of the LEC1 promoter, in accordance with the present invention, when operably linked to the LEC1 coding sequence, can cause high levels of expression of LEC1 in non-embryonic tissues, clearly demonstrating that the incomplete promoter fragment of the invention avoids the repressor mechanism which acts on the complete wild type LEC1 promoter. Without wishing to be bound by any particular theory, the present inventors hypothesise that the repressor mechanism acts on, or requires, that portion of the wild type LEC1 promoter which is deleted from the promoter fragment of the first aspect of the invention.

As noted in the prior art, overexpression of LEC1 in post-embryonic stages of development can cause activation of embryonic developmental pathways and formation of embryo-like morphological structures.

Typically, the expression of LEC1 in vegetative tissues in plants containing the isolated promoter fragment of the first aspect of the invention results in the production of plants wherein the hypocotyl has acquired embryonic traits. More typically, the plants exhibit a swollen hypocotyl due to a large accumulation of starch, storage lipids and protein post-embryonically.

For present purposes, the term “swollen hypocotyl” relates to an Arabidopsis seedling having a hypocotyl of at least 50% greater volume when compared with a wild type Arabidopsis seedling of the same age (e.g. in the range 7 to 14 days post germination). The volume of the hypocotyl can be estimated by simple microscopic examination.

Preferably, the isolated promoter fragment of the present invention comprises a deletion which causes a dominant mutation. In one embodiment, the dominant mutation is of low penetrance.

As explained above, in a second aspect, the present invention provides an isolated fragment of the LEC1 promoter comprising a deletion relative to the wild type LEC1 promoter, which isolated fragment possesses repressor activity in non-embryonic vegetative plant tissues, wherein the isolated fragment comprises at least 400 bases of the sequence shown in FIG. 1, or a functional equivalent thereof. By way of explanation, the portion which has been deleted from the wild type promoter in the first aspect of the present invention is the part of the LEC1 promoter which is required for lec to be repressed in non-embryonic tissues. Therefore, by extrapolation, the portion of the LEC1 promoter that has been deleted should have repressor activity.

Preferably, the isolated fragment of the second aspect of the invention, comprising a deletion relative to the wild type LEC1 promoter, comprises less than 3256 bases, more preferably less than 3200, and most preferably less than 3000 or 2500 bases of the sequence shown in FIG. 1, or a molecule of equivalent size exhibiting at least 95% sequence identity therewith.

In particular, the promoter fragment of the second aspect of the invention comprises an incomplete portion of the wild type LEC1 promoter. Preferably, the fragment of the second aspect of the invention comprises at least 500 bases, more preferably at least 1000 bases and most preferably at least 1500 bases. In particular, the promoter fragment of the second aspect of the invention may comprise up to 2000, 2500 or even up to 3000 bases.

In preferred embodiments, the promoter fragment of the second aspect of the invention will comprise a portion of at least 500 bases of the wild type complete LEC1 promoter sequence shown in FIG. 1, located between 436 and 3796 bases upstream of the promoter start site, or exhibits at least 95% sequence identity therewith.

Methods of manipulating DNA are known to those skilled in the art. The isolated fragment of the present invention can be made using standard recombinant methods, synthetic techniques, or combinations thereof. The reader is referred to Sambrook et al “Cloning. A Laboratory Manual”, 2^{nd} Edition, Cold Spring Harbor Press, New York (the content of which is herein specifically incorporated by reference).

In a third aspect, the invention provides a recombinant nucleic acid construct comprising an isolated promoter fragment, which fragment is in accordance with the first or second aspects of the invention defined above.

Conveniently, the construct may additionally comprise one or more of the following:—T-DNA to facilitate the introduction of the construct into plant cells; an origin of replication to allow the construct to be amplified in a suitable host cell; a nucleotide sequence encoding a polypeptide, which sequence is operably linked to the promoter fragment of the first or second aspect; a selectable marker (such as an antibiotic resistance gene); an enhancer element; and one or more further promoters, constitutive or inducible, which are preferably active in a suitable host cell, especially a plant host cell.

In a fourth aspect, the invention provides a method of causing transcription of a nucleic acid sequence, the method comprising the step of placing the sequence to be transcribed in operable linkage with an isolated promoter fragment in accordance with the first or second aspect of the invention. Preferably, the isolated fragment of the first or second aspect and the sequence to be transcribed are placed in operable linkage in a plant cell. The plant cell may be from a monocot or dicot plant and may, in particular, be from a plant which is a commercial source of starch, such as maize, potato, rice, cassava or the like.

The LEC1 mutant promoter drives the ectopic expression of the LEC1 gene in Arabidopsis, resulting in hyperaccumulation of starch and oil in tissues that normally accumulate very low levels. The use of the LEC gene family to increase oil yield in maize has been described (Allen et al, 2003 US 2003/0126638) and the content of that publication is specifically incorporated herein by reference. However, the inventors are not aware of any suggestion of the use of LEC1 and related genes as tools to increase starch production, which constitutes a preferred embodiment of the present invention. The present application particularly relates to the utility of LEC1 family genes for increasing starch yield in crops, including a ‘gene pyramiding’ approach of co-expression with genes affecting auxin synthesis or signalling.

The LEC1 gene is the first transcription factor to be identified that can switch on a starch biosynthetic pathway in vegetative tissues. This potentially could have an enormous impact on starch yield in crop plants. This discovery can be exploited in several ways, as described below.

1. The mutant Arabidopsis LEC1 promoter and gene could be introduced as a single entity into target crop species (e.g. potato, cassava, rice, maize etc) to induce the activation of starch accumulation in vegetative tissues, or in vitro cultured plant cells or tissues, that do not normally accumulate starch. Crop plant or cell culture transformation would be carried out by standard techniques such as Agrobacterium tumefaciens-mediated transformation, or direct gene transfer methods such as microprojectile bombardment (Casas et al, 1993, Proc. Natl. Acad. Sci. USA 90, 11212-11216); or protoplast transfection (Lindsey & Jones, 1988 New, Nucleic Acid Techniques, 519-536, Ed. J A Walker, Humana Press, Clifton, N.J.; Lindsey & Jones, 1989, Plant Cell Rep. 8, 71-74) known to those skilled in the art. To ensure maximal levels of effect, and in view of the promoting effect of auxin on starch accumulation mediated by the ectopic expression of LEC1, the LEC1 gene would preferably be co-transformed with a gene or genes designed to promote auxin accumulation or enhanced auxin sensitivity in LEC1-expressing cells. For example, one such gene is that encoding a predicted auxin receptor, Auxin Binding Protein 1 (ABP1), which confers enhanced auxin responsiveness to cells when over-expressed (Bauly et al 2000 Plant Physiol. 124, 1229-1238; Chen et al, 2001 Genes Devel. 15, 902-911). A second example of a gene to be used is the iaaM gene from Agrobacterium tumefaciens, encoding an auxin biosynthetic enzyme and which when over-expressed induces increased auxin accumulation and responses (Klee et al, 1987, Genes Devel. 1, 86-96). These examples are illustrative, and other genes affecting auxin synthesis, metabolism or cell sensitivity to auxin, known to those skilled in the art, could be used.

2. It is also possible to over-express directly in transgenic plants, or in vitro cultured plant cells or tissues, the LEC1 gene from Arabidopsis, or structurally and functionally related genes from target crop species, in concert with genes designed to modulate auxin synthesis, metabolism or cell sensitivity to auxin as illustrated above. A number of agronomically important plant species contain homologues of the Arabidopsis EC1 gene. Examples include Oryza sativa (rice, accession AY264284), Zea mays (maize, accession AF4101 76), Brassica napus (oilseed rape, accession CD814252), Helianthus annuus (sunflower, accession AJ879074) and Glycine max (soybean, accession AY058917). The homologues share between 35-60% identity to the LEC1 polypeptide over its entire sequence. It is likely that these homologues are functionally equivalent to the Arabidopsis LEC1 gene and therefore are attractive targets for the manipulation of starch deposition in vegetative tissue. Similar genes from other species could be isolated by standard molecular biology techniques known to those skilled in the art. For example, homologous genes could be isolated by degenerate PCR (Compton, 1990, PCR Protocols, pp. 39-45, Ed. Innis, Gelfand, Svinsky and White, Academic Press, New York); the screening of cDNA or genomic DNA libraries made from target crop species RNA by using Arabidopsis RNA or DNA sequences as probes; or the use of genomic or cDNA sequence information to design gene-specific PCR primers to allow the amplification and cloning of relevant genes or cDNAs. The sequences of the degenerate primers that may be used to amplify LEC1 homologues in plants are as follows:

### Forward Primer

(G/A)CA(A/G)GA(C/T)(C/A)(A/G)N(T/C)(A/T)(C/G/T)ATGCC(A/G)AT(C/A/T)G, or using standard IUPAC nomenclature, RCA RGA YMR NYW BAT GCC RAT HG

### Reverse Primer

C(G/C/A)(G/C)(T/C)(C/A)TC(T/A/G)A(T/A/G)(C/T)(C/T)C(A/C/G)C(G/T)(G/A)TA (C/G/A)C(G/T)(G/A/T) (or, using IUPAC nomenclature, CVS YMT CDA DYY CVC KRT AVC KD).

The above primers have the capacity to amplify a fragment of approximately 200 bp from agronomically important plant species. The amplified fragment could then be used as a probe against cDNA/genomic libraries. Alternatively, the technique of 5′ and 3′ RACE (rapid amplification of cDNA ends) would be required. As an alternative, the Arabidopsis LEC1 cDNA could be used as a probe under low stringency conditions. The full length LEC1 cDNA clone can be used for probing cDNA or genomic libraries. Appropriate amplification, isolation and screening techniques are well-known to those skilled in the art (e.g. in Sambrook et al, cited previously).

The expression of LEC1 or its homologues could be driven by either constitutive or widely-expressed promoters, such as the CaMV35S promoter, or others available to those skilled in the art; or gene promoters that would drive expression in specific tissues or organs.

3. It is possible to modify the promoters of LEC1 homologues to promote expression in vegetative tissues, and use these to drive expression of the LEC1 homologue in specific crop species. Deletion analysis of these promoters can be used to identify regions required for suppression of expression in vegetative tissues, using standard techniques known to those skilled in the art. Each deletion mutant homologous promoter and its gene could be introduced as a single entity into the respective target crop species to induce the activation of starch accumulation in vegetative tissues, or in vitro cultured plant cells or tissues, that do not normally accumulate starch.

4. Given the evidence that sucrose increases the starch accumulation phenotype of tissues ectopically expressing LEC1 in the presence of auxin, it may also be desirable to manipulate sucrose availability in transgenic tissues to maximise starch accumulation. In this example, sucrose concentration in cells could be increased by co-expression of sucrose transporters with LEC1 genes and auxin synthesis/signalling genes, as described above, with the aim of locally increasing LEC1 (or its homologue) expression, auxin responses and sucrose availability simultaneously. This represents a further ‘gene pyramiding’ to that described above.

In one embodiment, the isolated fragment of the first or second aspect of the invention and the sequence to be transcribed may be present on the same construct to be introduced into the plant cell. In another embodiment, the isolated fragment of the first or second aspect of the invention and the sequence to be transcribed are present on different constructs which are introduced into the plant cell such that the promoter and coding sequence are placed in operable linkage in a plant cell, typically following integration into the host cell genome. In a further embodiment, the sequence to be transcribed may be endogenous to the plant cell and introduction of the isolated fragment of the first or second aspect of the invention, and subsequent integration into the host cell genome sufficiently close to the target gene results in transcription of the endogenous sequence under the control of the promoter fragment of the first or second aspects of the invention.

In another embodiment, the presence of the sequence to be transcribed and/or the presence of the isolated fragment in accordance with the first or second aspect of the invention may be monitored using a reporter assay, wherein a reporter gene recognises the relevant sequences.

In the first aspect, the operable linkage of the sequence to be transcribed and the isolated fragment results in the upregulation of expression. In the second aspect, the operable linkage of the sequence to be transcribed and the isolated fragment results in downregulation of expression.

In some embodiments, promoter activity of the promoter fragment in accordance with the first or second aspect is regulatable by auxins, wherein the presence of auxin in a plant cell comprising the isolated promoter fragment causes expression of embryonic traits, such as accumulation of starch, lipid or protein. In a similar manner, the addition of sucrose to a plant cell comprising the isolated fragment of the first or second aspect enhances the penetrance of the embryonic phenotype. Conversely, the hormones gibberellin and abscisic acid (ABA) do not play a role in regulating the mutant phenotype, and cytokinin antagonises the penetrance of the mutant phenotype. Thus, for example, the role of LEC1 in the control of embryonic cell fate may require the presence of auxin and sucrose to promote cell division and differentiation.

In a fifth aspect, the present invention provides an altered plant, wherein the isolated promoter fragment in accordance with the first or second aspect has been introduced into a plant cell and wherein a plantlet is subsequently produced from the cell, or wherein the sequence has been introduced into a plant. The invention also provides the progeny of such a plant or plantlet, which progeny retain the introduced promoter fragment, preferably in a stable manner (i.e. pass on the relevant nucleic acid molecule to their own progeny).

Examples of a plant that may be transformed according to the method of the present invention, but are not limited to, Arabidopsis thaliana (Columbia-O ecotype), and also include monocotyledonous and dicotyledonous plants such as maize, wheat, rice, barley, oats, soybean, cassava, turnip and swede. Methods of transforming monocotyledonous and dicotyledonous plants are know to those skilled in the art and include, for example, techniques such as electroporation, particle bombardment, microinjection of plant cell protoplasts or embryonic callus, Agrobacterium tumefaciens-mediated transformation techniques and gene gun techniques. Particularly preferred are those plants used commercially as sources of starch, such as maize and cassava.

The invention also provides a method of altering a plant, the method comprising the introduction into the plant of an isolated fragment in accordance with the first or second aspect of the invention, and/or introduction of a nucleic acid construct in accordance with the third aspect of the invention. The invention also provides a method of altering a plant by introduction of the promoter fragment of the first or second aspect, or a construct in accordance with the third aspect, and optionally generating a plantlet and/or plant from the transformed plant cell. Preferably the plant will be altered so as to possess a desirable trait, such as increased storage of starch or other storage molecules in vegetative tissue. If desired, one or more starch synthesis enzymes (e.g. soluble starch synthase, granule bound starch synthase, starch branching enzymes) in the plant could be upregulated or modified in the same plant in order to increase or alter starch synthesis.

The isolated fragment of the present invention was identified using the technique of gene tagging in Arabidopsis thaliana plants. Essentially, the present inventors carried out a screen for mutants of Arabidopsis that exhibited embryonic characteristics in post-embryonic seedlings. Specifically, the inventors carried out a screen for mutations that caused a modification of the expression pattern of a molecular marker of embryonic and seedling polarity, the POLARIS (PLS) gene. The PLS gene was first identified by a promoter trap, exhibiting GUS expression in the basal region of the heart-stage embryo of Arabidopsis thaliana plants (Topping et al, 1994 Plant J. 5, 895-903; Topping and Lindsey, 1997 Plant Cell 9, 1713-1725). The PLS gene encodes a peptide comprising a predicted 36 amino acid residues which is required for correct hormone signalling and development (Casson et al, 2002 Plant Cell 14, 1705-1721).

The present inventors transformed a large population of the PLS-GUS promoter trap line of Arabidopsis with T-DNA from Agrobacterium tumefaciens. The inventors found that a likely aborted T-DNA insertion event led to a deletion mutation close to the LEC1 native gene, which in turn led to the expression of that gene in vegetative tissues. The mutation is a gain-of-function mutation. Such a mutation would modify the expression of the LEC1 gene by activating an embryonic pathway in vegetative tissues. The mutation identified by the present inventors was designated the “turnip” (tnp) mutation.

For the avoidance of doubt, it is hereby expressly stated that any feature of the invention described as “preferable”, “preferred”, “advantageous”, “desirable” or the like may be present in isolation or combined with any other feature or features so described, unless the context dictates otherwise.

The following Examples illustrate, but do not limit, the invention. The Examples refer to drawings in which:

FIG. 1 shows the genomic sequence of the LEC1 coding sequence from Arabidopsis thaliana (Col-O ecotype) in which the first and second exons are underlined and the alternative ATG ‘start’ codons are boxed, together with some untranscribed sequence upstream therefrom;

FIG. 2 shows the sequence of the wild type LEC1 promoter from Arabidopsis thaliana, wherein that portion which may be deleted (in some embodiments of the promoter fragment of the invention) is underlined;

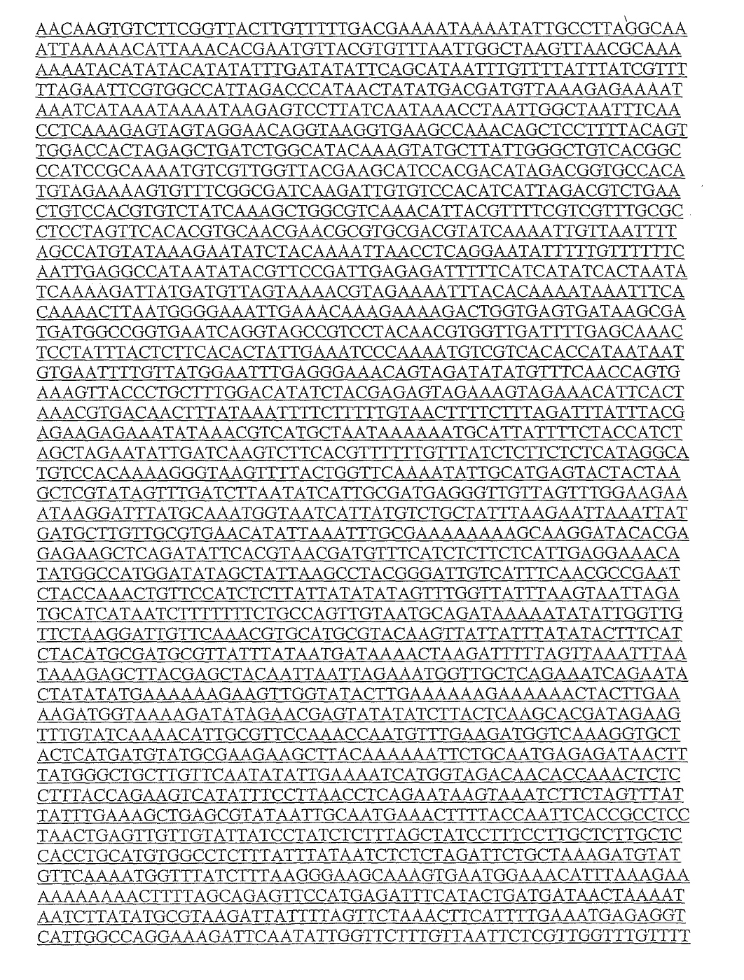

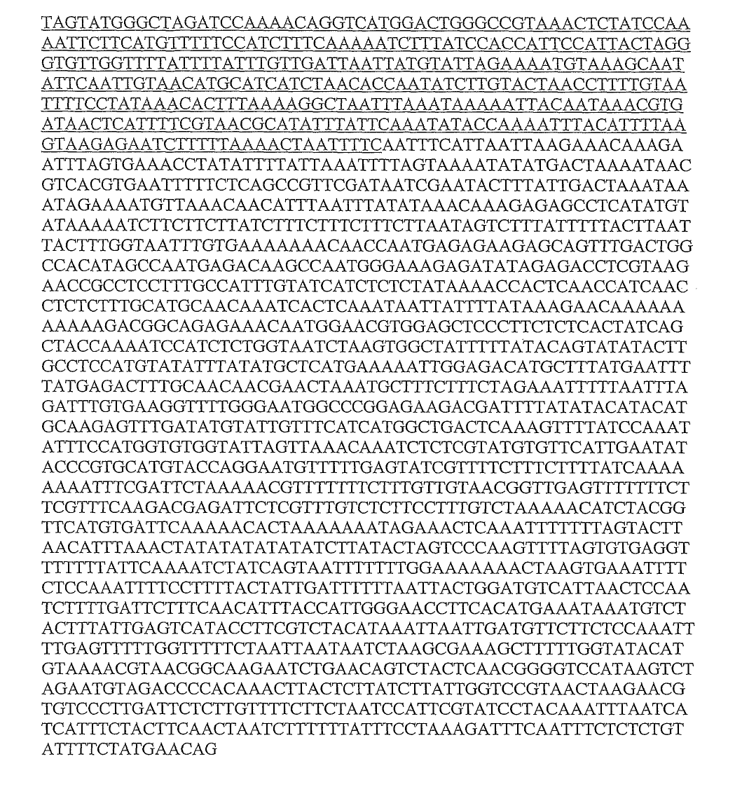

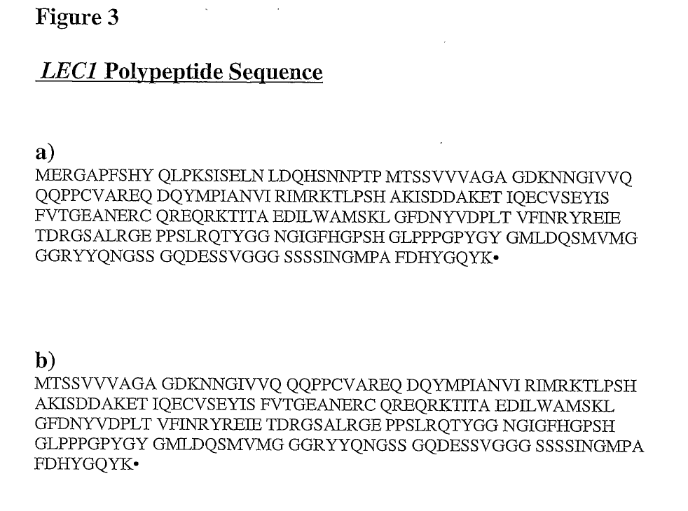

FIGS. 3a) and b) show the variants of the LEC1 gene containing the first and second exons as shown in FIG. 1;

FIG. 4 illustrates the phenotype of the tnp Arabidopsis seedlings;

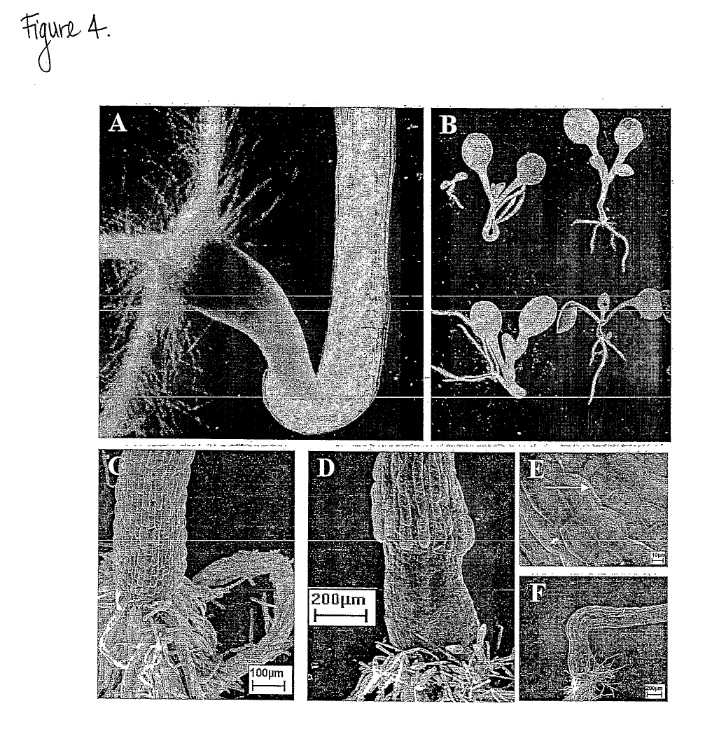

FIG. 5 illustrates the phenotype of the original tnp mutant (A) and primary transformants containing the tnp locus (B+C), wherein the arrows indicate tnp-like hypocotyls;

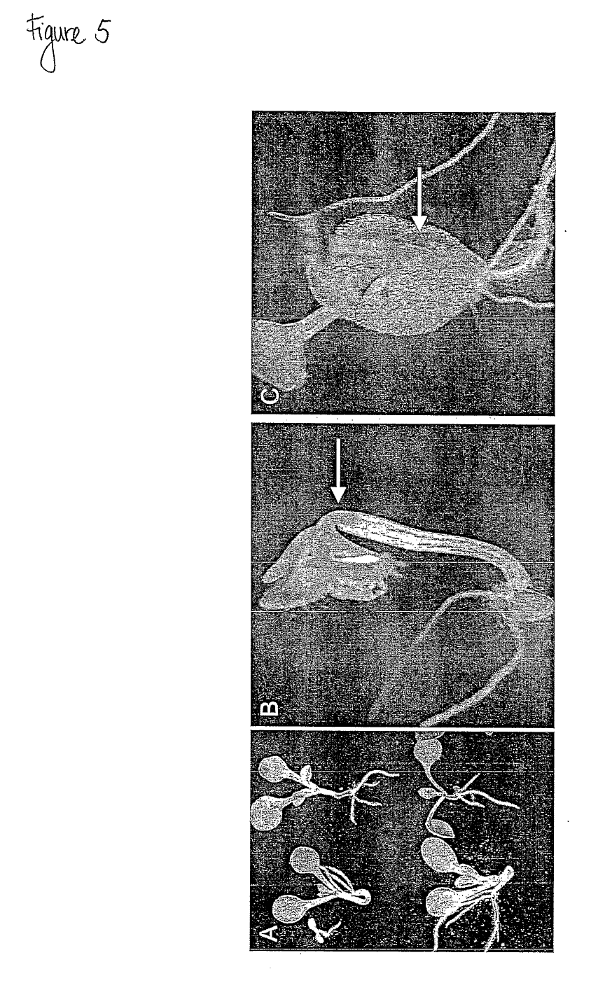

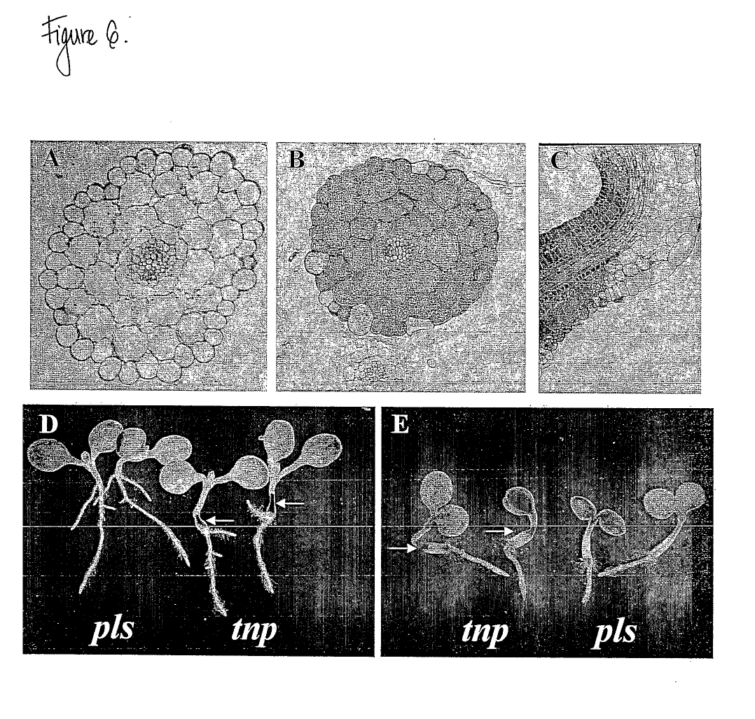

FIG. 6 provides an illustration of the accumulation of storage compounds in tnp Arabidopsis seedlings;

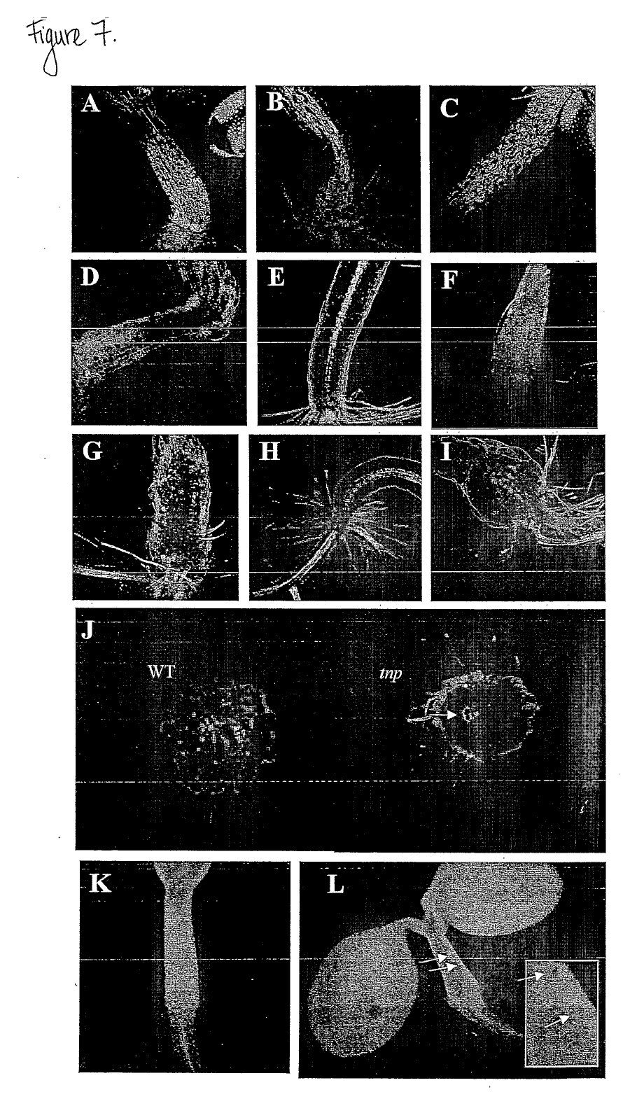

FIG. 7 provides an analysis of the level of gene expression in a tnp mutant;

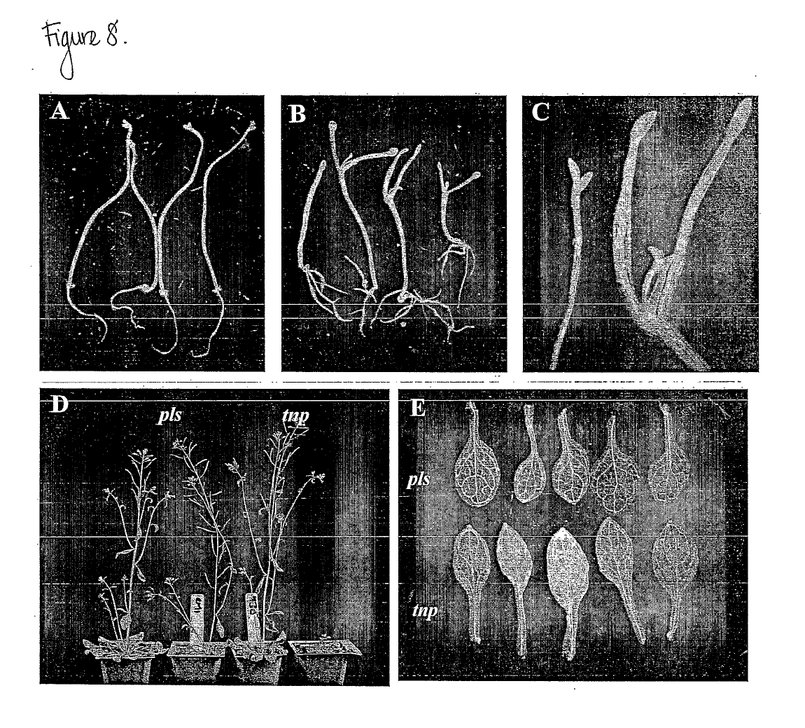

FIG. 8 illustrates that tnp mutants are defective in other aspects of development;

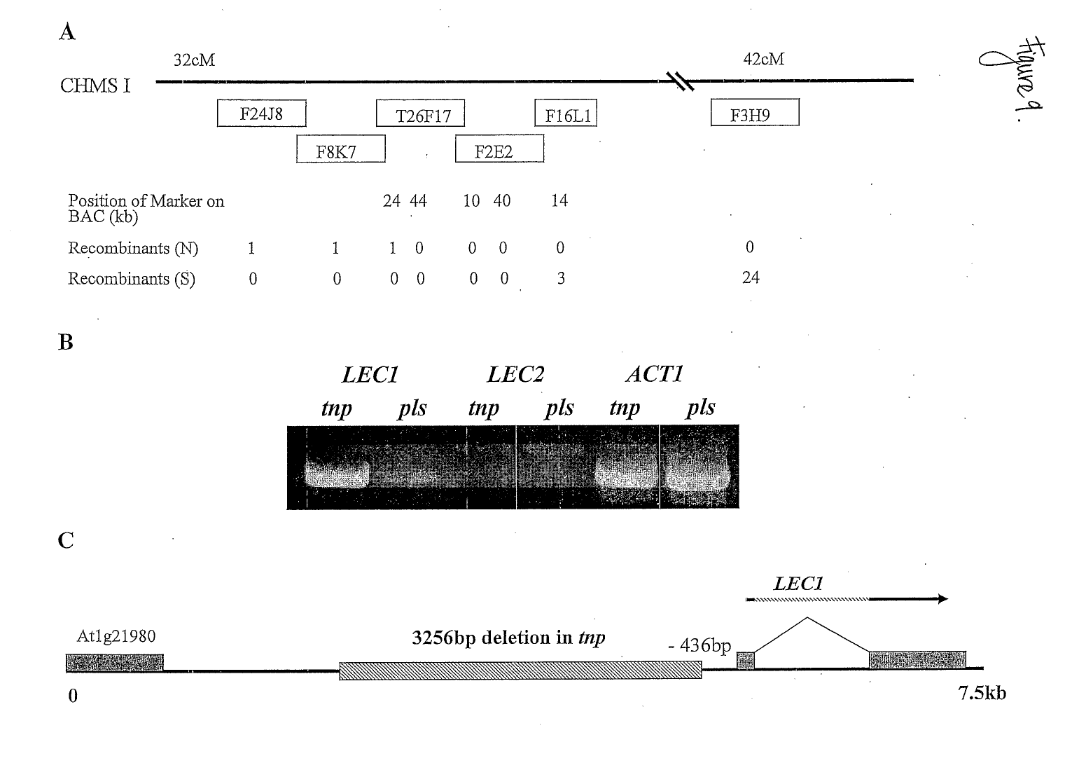

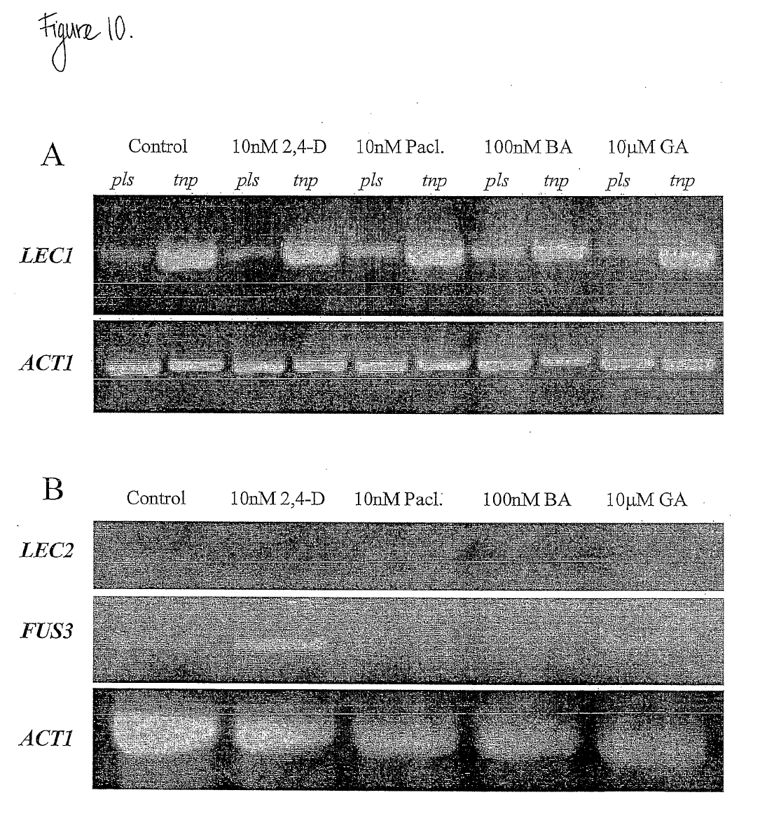

FIG. 9 is a schematic illustration of the LEC1 gene, showing the localisation of the TNP locus;

FIG. 10 shows the effect of plant growth regulators on gene expression in the tip mutant; and

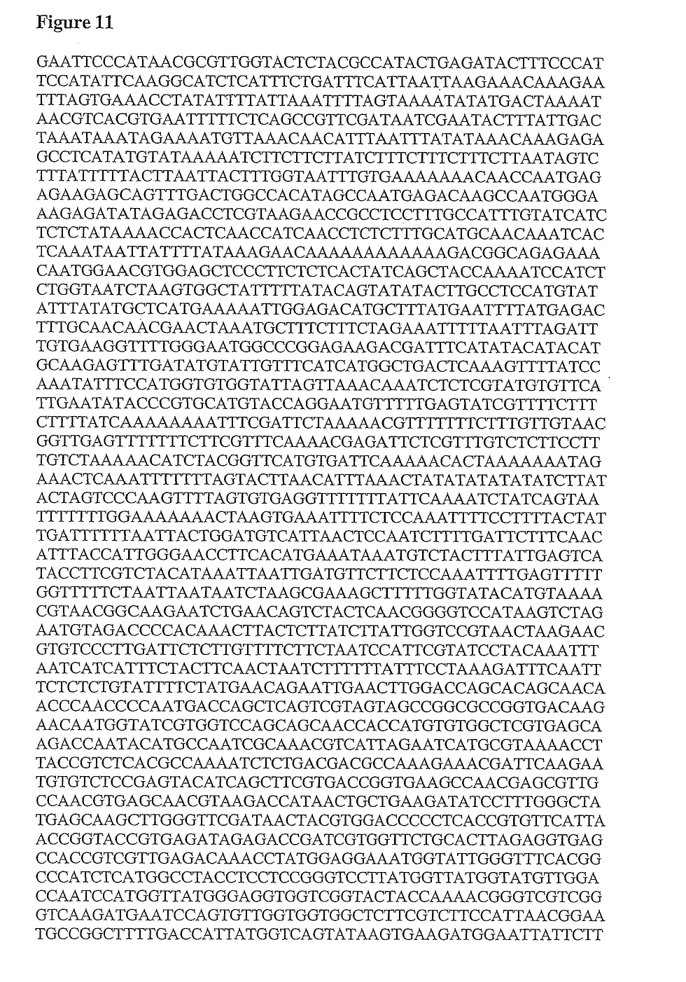

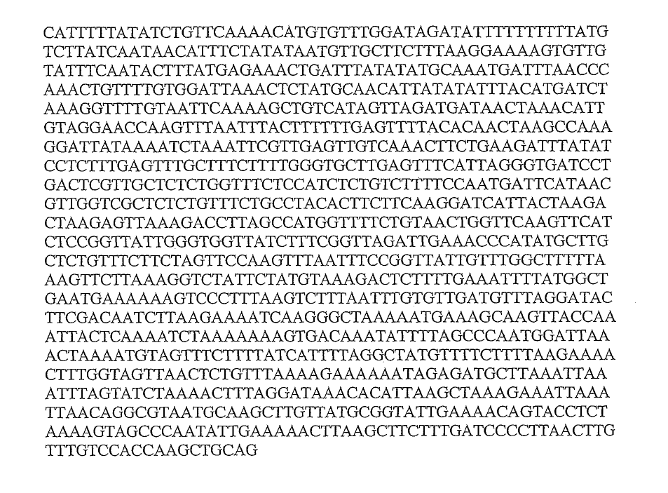

FIG. 11 shows the sequence of the tnp locus used for reiteration of the tnp mutant phenotype.

### EXAMPLES

### Example 1

### Identification of the Turnip (tnp) Mutant

Plants containing the tnp mutation were isolated in an activation-tagging screen of pls mutants which were defective in a gene encoding a predicted small polypeptide necessary for correct root growth (Casson et al, 2002, The Plant Cell 14, 1705-1721). In order to identify modifiers of PLS expression, plants from the pls line (Arabidopsis thaliana ecotype C24, containing the promoter trap p gusBin19, Topping et al, 1991, Development 112, 1009-1019; Topping et al, 1994, Plant J. 5, 895-903; Casson et al 2002, Plant Cell 14, 1705-1721) were transformed with the activation tag construct, consisting of a tandem repeat of 4×CaMV35S enhancer elements in the binary vector pMOG 1006 (Mogen, Leiden, The Netherlands). Plants were transformed by the floral dip method (Clough and Bent, 1998, Plant J. 16, 735-743) using Agrobacterium tumefaciens C58C1 (Dale et al, 1989, Plant Sci. 63, 237-245). The transgenic population was screened for mutants in which β-glucuronidase (GUS) expression had been altered. Of the lines that were screened, line number 930 showed abnormal expression of PLS-GUS at the junction between the hypocotyl and root of the plant (see FIG. 3a). A swollen and dense structure was formed at this position. This phenotype segregated and was called the “turnip” (tnp) mutant.

### Example 2

### The tnp Mutation is Dominant but Shows Incomplete Penetrance

The number of tnp seedlings present in the T2 population was greater than would have been expected for a single, recessive locus, thus suggesting that the tnp mutation is dominant. In particular, whereas the T2 population was found to contain 126 wild type plants, as many as 170 plants were found to contain the tnp mutation. Segregation analysis of T2 seedlings revealed that the tnp mutation was not linked to the insertion of T-DNA. The technique of PCR was used to analyse the F2 progeny of plants produced following crossing of wild-type Arabidopsis thaliana (Col-O) with plants containing the tnp mutation. The results indicated that the mutation was not due to the presence of a partial activation tag, and was not dependent on the pls mutation (data not shown).

Although the experimental data indicated that the tnp mutation was dominant, analysis of independent T3 lines showed that the penetrance of the tnp phenotype varied significantly between lines, with values ranging from approximately 0 to 60%. In order to establish whether the incomplete penetrance was due to methylation-dependent gene silencing, individual T3 sibling lines were germinated in the presence of 100 μM 5-azacytidine, a methylation inhibitor (Jones and Taylor, 1980 Cell 20, 85-93). The addition of 5-azacytidine to each line caused an increase in the penetrance of the tnp phenotype when compared with control levels, although the effect was found to be highly variable between independent lines (see Table 1). However, the results suggested that methylation-mediated gene silencing was partially responsible for the incomplete penetrance of tnp.

TABLE 1

| T3 Line | Medium | TNP | Tnp | % tnp |
|---|---|---|---|---|
| 1 | 1/2MS10 | 53 | 21 | 28 |
| 1 | 100 μm 5-AZA-C | 30 | 49 | 62 |
| 7 | 1/2MS10 | 120 | 0 | 0 |
| 7 | 100 μM 5-AZA-C | 112 | 9 | 7.4 |
| 21 | 1/2MS10 | 75 | 52 | 41 |
| 21 | 100 μM 5-AZA-C | 71 | 65 | 48 |

### Example 3

### The tnp Mutant Shows an Altered Cell Identity

Seedlings containing the tnp mutation exhibited a high degree of phenotypic variability. On rare occasions, the tnp mutation was lethal to the seedlings (see FIG. 4b). Examination of embryos from tip and control siliques did not reveal any morphological differences, suggesting that the phenotypic defect developed after germination. The technique of scanning electron microscopy was used to investigate the surface patterning of the abnormal hypocotyl. The epidermal cells were much smaller and flatter than those of the pls parent (see FIGS. 4c and 4d). The cells containing the tnp mutation remained in strict files. However, abnormal cell division occasionally occurred within a file wherein the mutation resulted in the generation of a number of cells that were small in size (see FIG. 4e). At the boundary of abnormal cell division, the cells were seen to undergo excessive elongation (FIG. 4f). In an attempt to determine whether this altered morphogenesis was associated with a change in the internal cell patterning, radial and longitudinal sections of the structure were examined. No obvious patterning defects were observed (see FIGS. 6a and 6b). However, sectioning revealed that the cells in the abnormal region of the hypocotyl were virtually devoid of a vacuole, and that the transition from abnormal to normal cells did not occur at a strict boundary across the structure (see FIG. 6c).

The absence of a large central vacuole and the dense staining of cells with the dye toluidine blue led to the proposal that the cells had formed a storage tissue. The cells were therefore tested for the presence of storage compounds. Staining with Lugol's solution indicated that the cells contained starch granules (FIG. 6d). Furthermore, staining with the dye Fat Red suggested the presence of a large amount of triacylglycerols (see FIG. 6e).

The alteration of cell morphogenesis, accumulation of high levels of starch and triacylglycerols and altered expression of PLS in the abnormal hypocotyl region led to the suggestion that the identity of the cells had changed. To farther investigate this hypothesis, the expression pattern of other markers was monitored. The epidermal cells of the hypocotyl are marked by expression of the Haseloff J2662 and J2601 GFP marker lines (http://www.plantsci.cam.ac.uk/Haseloff). In seedlings containing the tnp mutation, expression was absent in cells of the abnormal hypocotyl, but was present in those cells above the hypocotyl (see FIG. 7a-d). In addition, the ARR5/IBC6::GFP marker (Brandstatter and Kieber, 1998 Plant Cell 10, 1009-1019; Casson et al, 2002 Plant Cell 14, 1705-1721) is normally expressed in pericycle cells of the root and hypocotyl and is also a marker of cytokinin responsiveness. However, in tnp seedlings the expression was found to be highly variable both in the abnormal hypocotyl and in morphologically normal hypocotyl cells, most often appearing in the epidermal cell layer (FIG. 7e-g). The expression of a SCR::GFP marker (Wysocka-Diller et al, 2000 Development 127, 595-603) was used to examine endodermal cell identity. Although expression was evident in the root and morphologically normal hypocotyl cells, the expression was virtually absent in the abnormal structure. The analysis of transverse sections of the hypocotyl of seedlings containing the tnp mutation did however reveal that rare, vacuolated cells showed SCR::GFP expression (see FIGS. 7h-j). Ordinarily, growth of the hypocotyl following embryogenesis occurs via cell expansion. In order to examine whether this was the case in tnp seedlings, a CYCAT1:CDB:GUS marker (Hauser and Benfey, 2000 Plant and Soil 226, 1-10) was used to examine cell division events. As expected, no cell division was observed in wild-type seedlings, whereas cell division was evident in seedlings containing the tnp mutation (see FIGS. 7k-l).

### Example 4

### Seedlings Containing the tnp Mutation Exhibit Defective Growth in Dark Conditions

The growth of seedlings containing the tnp mutation in the dark and in the presence of 1% sucrose resulted in a lower rate of penetrance than was observed in light-grown tnp seedlings (i.e. having a penetrance of 11.3%±SE in dark growth conditions versus 16.7%±SE in light growth conditions). In addition, experiments also showed that seedlings grown in the dark underwent partial de-etiolation (see FIG. 8a-c). During growth in dark conditions, the shoot apical meristem (SAM) of the pls control plants did not develop. In contrast, during growth of seedlings containing the tnp mutation, the petioles of the cotyledons expanded and the first leaves developed after seven days. Previous experiments have shown that contact of the SAM with a sucrose-containing medium gives rise to a similar effect (Roldin et al, 1999 Plant J. 20, 581-590). The present inventors observed that in 60% of tnp seedlings showing partial de-etiolation, the SAM was not in contact with the growth medium. However, the de-etiolated phenotype was shown to be more pronounced in those seedlings that did show contact. In addition to differences in the activity of SAM, the root system of tnp seedlings was also different to that of pls, having a greater number of, and more elongated, lateral roots (see FIGS. 8a-b). In contrast, there was no difference in the root architecture of light-grown pls and tnp seedlings (data not shown).

Other aspects of development were also affected in plants containing the tnp mutation. In particular, flowering time was found to be highly variable. Although the majority of plants flowered at the same time as the pls parental line, some tnp plants were found to be late flowering (see FIG. 8d). Examination of the first true leaves of tnp plants revealed that they were more elliptical than those of pls plants (see FIG. 8e).

### Example 5

### Cloning of TNP

Segregation analysis showed that the tnp mutation was not associated with the insertion of T-DNA. A map-based cloning strategy was therefore used wherein an F2 mapping population was generated by outcrossing plants containing the tnp mutation (C24-ecotype) with the Col-O ecotype of Arabidopsis thaliana. The TNP locus was tentatively positioned at approximately 40 cM on chromosome I using the simple sequence length polymorphism (SSLP) marker nga 280 (83 cM).

In order to further map the TNP locus, a strategy was developed that would account for the dominant phenotype of tnp linked with the incomplete penetrance. Seeds from the F2 mapping population were germinated on medium containing 20% sucrose and 25 nM 2,4-dichlorophenoxyacetic acid (2,4-D), which had been found to result in the highest penetrance of tnp without having an effect on growth, thus increasing the proportion of TNP/tnp heterozygotes in the population. SSLP analysis was then performed, with the markers expected to be located on either side of the TNP gene. Plants were identified that had a Col-O ecotype at one marker and a Col-O/C24 ecotype at the second marker, and vice versa. Thus, the dominant tip heterozygote was used to map the locus of TNP. Using this approach, 24/800 plants were found to be Col-O with the marker nga 248 (42.17 cM, BACF3H9) and 1/800 plants was Col-O at the marker F24J8 (approximately 32 cM, BAC F24J8). These plants were Col-O/C24 heterozygotes at the alternative marker. Fine mapping led to the determination that TNP was located at either the BAC T26F17 or F2E2 locus (see FIG. 9a).

BAC T26F17 contains the LEC1 gene (Lotan et al, 1998 Cell 93, 1195-1205) which is expressed ectopically after germination in the pkl mutant (Ogas et al, 1999 Proc. Natl. Acad. Sci. USA 96, 19839-19844). The pkl root phenotype is reminiscent of the tnp hypocotyl phenotype, thus suggesting that the LEC1 gene was a potential candidate for TNP. Therefore, the genomic region containing the LEC1 coding sequence was amplified from tnp mutants and sequenced, but no nucleotide differences were identified between the tnp and pls parental lines. One explanation for this observation is that a nucleotide change in the LEC1 promoter may result in the expression of LEC1 in vegetative tissues, as observed in the pkl mutant. Therefore, semi-quantitative reverse transcriptase (RT)-PCR experiments were performed to determine the levels of LEC1 transcript in seedlings at 1 to 2 days post-germination. Although low levels of LEC1 were detected in RNA from control germinating seedlings, the LEC1 transcript levels were strongly upregulated in the tnp mutant. In contrast, LEC2 which is also upregulated in pkl (Dean Rider et al, 2003 Plant J. 35, 33-43), remained unaffected (see FIG. 9b).

To determine whether the upregulation of LEC1 in tip seedlings was due to a mutation in the promoter region, genomic DNA upstream of the LEC1 transcriptional start site was amplified by thermal asymmetric interlaced (TAIL) PCR (Liu et al, 1995 Plant J. 8, 457-463) and sequenced. These results revealed that tnp seedlings contained a deletion of 3256 bp, at a site approximately 436 base pairs upstream of the putative LEC1 transcriptional start site (FIG. 9c). PCR analysis of the F2 generation of tnp seedlings from the mapping population showed that the deletion was present in all plants tested, thus suggesting that this deletion is responsible for the tnp phenotype.

### Example 6

### Auxin and Sucrose Increase Penetrance of the tnp Phenotype

The tnp mutant phenotype is similar to that of the pkl mutant, which is characterised by the development of swollen and greenish roots that accumulate triacylglycerols and protein bodies (Ogas et al, 1997 Science 277, 91-94; Ogas et al, 1999 Proc. Natl. Acad. Sci. USA 96, 13839-13844). The penetrance of the pkl mutant phenotype is also variable and is affected by gibberellic acid and the gibberellin biosynthesis inhibitor, uniconazole-P. In order to determine whether growth factors have an effect on the penetrance of the tnp phenotype, the tnp seed was germinated and grown in the presence of a number of compounds (see Table 2).

TABLE 2

| Medium | | TNP | Tnp | % tnp |
|---|---|---|---|---|
| 1/2 MS10 | | 162 | 79 | 32.8 |
| NAA 10 | nM | 83 | 62 | 42.8 |
| IAA 50 | nM | 75 | 168 | 69.1 |
| 2,4-D 10 | nM | 34 | 121 | 78.1 |
| NPA 10 | uM | 86 | 107 | 55.4 |
| NOA 10 | uM | 61 | 87 | 58.8 |
| BA 100 | nM | 91 | 10 | 9.9 |
| ACC 100 | pM | 104 | 43 | 29.3 |
| GA 10 | uM | 96 | 57 | 37.3 |
| 1/2MS10 | | 145 | 133 | 47.8 |
| GA 10 | nM | 129 | 101 | 42.1 |
| GA 100 | nM | 123 | 133 | 52 |
| GA 1 | μM | 117 | 164 | 58.4 |
| GA 10 | μM | 107 | 89 | 45.4 |
| Paclobutrazol 10 | nM | 32 | 178 | 84.7 |
| Paclobutrazol 100 | nM* | 13* | 34* | 72.3* |
| BA 10 | nM | 153 | 187 | 55 |
| BA 100 | nM | 203 | 112 | 35.6 |
| BA 1 | μM | 304 | 16 | 5 |
| 2,4-D 10 | nM | 42 | 192 | 82.1 |
| 2,4-D 100 | nM | 9 | 183 | 95.3 |
| 2,4-D 1 | μM | 4 | 224 | 98.2 |

As observed with pkl, the penetrance of tip was increased in the presence of a gibberellic acid biosynthetic inhibitor, known as paclobutrazol. However, unlike pkl, gibberellic acid did not suppress the penetrance of tnp and was only shown to have a weakly positive effect on tnp. Interestingly, when the tnp seed was germinated in the presence of both 10 nM paclobutrazol and 10 μM gibberellic acid (GA), the positive effect of the paclobutrazol was partially suppressed (data not shown).

The natural auxin indole-3-acetic acid (IAA) and synthetic auxins naphthaleneacetic acid (NAA) and 2,4-D each had a positive effect on the penetrance of the tnp phenotype at low concentration. Of the auxins tested, 2,4-D was the most effective, increasing penetrance to nearly 100% at a concentration of 1 μM. The auxin transport inhibitors 1-N-naphthylphthalamic acid (NPA) and naphthoxyacetic acid (NOA) were also found to have a positive effect on tnp penetrance, whereas the ethylene precursor 1-aminocyclopropane-1-carboxylate (ACC) had little effect. The cytokinin N(6)-benzyladenine (BA) was the only compound tested that markedly suppressed penetrance of the tnp phenotype, although this was only significant at concentrations above 100 nM. Amongst other compounds tested, abscisic acid (ABA) was not found to have an effect on penetrance and tnp seedlings showed no difference in sensitivity to ABA in germination studies (data not shown).

Given the large quantities of starch stored in tnp, the effect of sugars on penetrance was examined. The absence of sucrose in the medium resulted in a complete loss of penetrance of the tnp phenotype, whilst the highest level of penetrance was observed with a concentration of 2% sucrose. The addition of 10 nM 2,4-D to the medium resulted in greater penetrance than with sucrose alone, even in the absence of sucrose suggesting that these compounds act via different pathways to increase penetrance (Table 3). Furthermore, the effect of 1% glucose or fructose was not as potent as the effect obtained from sucrose alone. However, the addition of 2,4-D resulted in comparable rates of penetrance, thus indicating that auxin is more effective at increasing the penetrance than is the carbon source from sugars (data not shown). The effect of sugars on the penetrance of tnp was not due to an osmotic effect, since mannitol was ineffective at inducing penetrance in the absence of sugar (data not shown).

TABLE 3

| Medium | TNP | tnp | % tnp |
|---|---|---|---|
| 0% suc. | 184 | 0 | 0 |
| 0% suc. + 10 nm 2,4-D | 138 | 3 | 2 |
| 1% suc. | 70 | 107 | 60.5 |
| 1% suc. + 10 nm 2,4-D | 21 | 120 | 85.1 |
| 2% suc. | 53 | 100 | 65.4 |
| 2% suc. + 10 nm 2,4-D | 13 | 155 | 92.3 |
| 3% suc. | 64 | 90 | 58.4 |
| 3% suc. + 10 nm 2,4-D | 23 | 122 | 84.1 |
| 4% suc. | 78 | 86 | 52.4 |
| 4% suc. + 10 nm 2,4-D | 46 | 102 | 68.9 |
| 5% suc. | 79 | 93 | 54.1 |
| 5% suc. + 10 nm 2,4-D | 28 | 131 | 82.4 |
| 6% suc. | 87 | 65 | 42.8 |
| 6% suc. + 10 nm 2,4-D | 50 | 97 | 66.0 |

High concentrations of sucrose or glucose have been shown to inhibit germination and have been used in selection screens for identifying sugar sensitive mutants (Laby et al, 2000 Plant J. 23, 587-596). In order to determine whether the effect of sugars on tnp penetrance was due to a change in sugar sensitivity, the effect of high sucrose or glucose concentrations on germination was examined. No difference between pls and tnp mutants was observed in the presence of 1-10% sucrose or 7% glucose (data not shown), thus suggesting that tnp is not hypersensitive or insensitive to these sugars.

### Example 7

### Auxin, Paclobutrazol and Cytokinin Do Not Affect LEC1 Transcript Levels

It is possible that auxin, paclobutrazol and cytokinin may affect tnp penetrance by altering the level of the LEC1 transcript, with a higher transcript level being associated with greater penetrance. To investigate the mechanism by which these compounds work, germinating seedlings were treated with each of the compounds, as well as with gibberellic acid (which has no effect on penetrance), and RNA was extracted at 1-2 days post-germination. LEC1transcript levels were determined by semi-quantitative RT-PCR and were found to be unaltered in response to these compounds in both tnp and pls controls (see FIG. 10a). Therefore, the effect of these compounds on penetrance is not mediated by alterations in LEC1 transcript levels, although post-transcriptional or translational effects cannot be excluded.

Alternatively, it is possible that these compounds may act by changing the expression of other key embryonic regulators to alter penetrance. FUS3 and LEC2 play key roles in embryogenesis and the transition to germination (Luerben et al, 1998 Plant J. 15, 755-764; Stone et al, 2001 Proc. Natl. Acad. Sci. USA 98, 11806-11811; Kroj et al, 2003 Development 130, 6065-6073). Semi-quantitative RT-PCR was used to determine whether the transcript levels in these genes were affected in tnp seedlings at 1-2 days post-germination in response to treatment with these compounds (see FIG. 10b). Treatment with 2,4-D, BA or gibberellic acid was found to significantly reduce the levels of LEC2 transcripts in tnp seedlings, whereas paclobutrazol had no effect. In the case of FUS3, treatment with 2,4-D was found to increase the transcript levels, whereas treatment with BA caused a reduction in the levels. Treatment with paclobutrazol and gibberellic acid did not have a significant effect on FUS3 transcript levels in the tnp mutant.

### Example 8

In the turnip (tnp) mutant a deletion in the promoter of the LEC1 gene is proposed to result in ectopic LEC1 expression in vegetative tissue resulting in a phenotype that includes conversion of the hypocotyls into a modified storage organ containing increased amounts of starch.

As a proof of principle, the complete mutant tnp genomic locus, a 3.4 kb fragment comprising the deleted promoter and the complete LEC1 coding sequence, was amplified from tnp mutants by polymerase chain reaction using the oligonucleotides TNPlocusFor3 (GAATTCCCATAACGCGTTGGTACTCTACGC) and TNPlocusRev3 (CTGCAGCTTGGTGGACAAACAAGTTAAGGG). This region was cloned into the binary vector pCIRCE and was then transformed into wild-type Arabidopsis (Col-O ecotype) by Agrobacterium mediated transformation. Primary transformants were identified by their resistance to the antibiotic kanamycin as conferred by the pCIRCE T-DNA. Amongst these primary transformants were seedlings resembling the original tnp mutant indicating that the presence of the mutant tnp locus is sufficient to induce the phenotypic alterations observed in the original tnp line.

1. An isolated fragment of the LEC1 promoter comprising a deletion, relative to the wild type LEC1 promoter, which isolated fragment possesses promoter activity in non-embryonic vegetative plant tissues in Arabidopsis, wherein the isolated fragment comprises at least 500 bases of the sequence shown in FIG. 1, or a functional equivalent thereof, which functional equivalent also possesses promoter activity in non-embryonic vegetative plant tissues of Arabidopsis and which exhibits 95% sequence identity over a portion of at least 500 bases of sequence as shown in FIG. 1 as determined by the sequence alignment program CLUSTALW (Chenna et al, 2003, Nucleic Acids Res 31, 3497-3500).

2-43. (canceled)

44. An isolated fragment of the LEC1 promoter according to claims 1, wherein the isolated fragment causes, in non-embryonic tissue, at least 50% of the level of expression caused by the same construct in embryos.

45. An isolated fragment of the LEC1 promoter according to claim 1, wherein there is deleted from the promoter fragment, relative to the complete wild type LEC1 promoter, at least 1000 bases.

46. An isolated fragment of the LEC1 promoter according to claim 1, wherein the portion which is deleted from, and therefore absent from, the promoter fragment comprises the portion of the sequence located at position −1500 to −2000 of the full length promoter, wherein position −1 is the first base upstream from the start codon, and wherein the start codon may be either of the ATG codons shown boxed in FIG. 1.

47. An isolated fragment of the LEC1 promoter fragment according to claim 1, wherein the portion of the wild type promoter which is deleted from the promoter fragment comprises the portion of the sequence located at position −436 to −3792 of the full length promoter, wherein position −1 is the first base upstream from the start codon, and wherein the start codon may be either of the ATG codons shown boxed in FIG. 1.

48. An isolated fragment according to claim 1, wherein the fragment is operably linked to a structural coding sequence, such that when present in a non-embryonic plant cell, the coding sequence is expressed.

49. An isolated fragment according to claim 48, wherein the coding sequence is a LEC1 coding sequence encoding a polypeptide which has 90% sequence identity to the wild type LEC1 coding sequence shown in FIG. 2.

50. An isolated fragment of the LEC1 promoter comprising a deletion, relative to the wild type LEC1 promoter, which isolated fragment possesses repressor activity in non-embryonic vegetative plant tissues, wherein the isolated fragment comprises at least 400 bases of the sequence shown in FIG. 1, or a functional equivalent thereof, which functional equivalent exhibits at least 95% sequence identity over a portion of 400 bases.

51. An isolated fragment of the LEC1 promoter according to claim 50, wherein the fragment comprises at least 1000 bases.

52. An isolated fragment of the LEC1 promoter according to claim 50, wherein the fragment comprises at least 1500 bases.

53. An isolated fragment of the LEC1 promoter according to claim 50, wherein the fragment comprises a portion of at least 500 bases of the wild type complete LEC1 promoter sequence shown in FIG. 1, located between 436 and 3796 bases upstream of the promoter start site, or exhibits at least 95% sequence identity therewith.

54. An isolated fragment of the LEC 1 promoter according to claim I in operable linkage with LEC1 which, when expressed in a plant, causes the plant to acquire embryonic traits and causes the accumulation of starch and/or oil or fatty acids and the like in vegetative tissues.

55. A nucleic acid construct comprising an isolated promoter fragment in accordance with claim 1.

56. A nucleic acid construct in accordance with claim 55, wherein the construct comprises a promoter fragment operably linked to a coding sequence.

57. A nucleic acid construct in accordance with claim 56, wherein the coding sequence is a LEC1 coding sequence.

58. A nucleic acid construct in accordance with claim 56, wherein the construct comprises one or more of the following: T-DNA to facilitate the introduction of the construct into plant cells; an origin of replication to allow the construct to be amplified in a suitable host cell; a nucleic acid sequence encoding a polypeptide, which sequence is operably linked to the promoter fragment of claim 1; a selectable marker (such as an antibiotic resistance gene); an enhancer element; and one or more further promoters, constitutive or inducible, which are active in a suitable host cell.

59. A method of causing transcription of a nucleic acid sequence, wherein the method comprises the step of placing the sequence to be transcribed in operable linkage with an isolated fragment in accordance with claim 1, and causing transcription of the sequence under the control of the promoter fragment in a suitable host cell.

60. The method of claim 59, wherein the isolated fragment of claim 1 and the sequence to be transcribed are present on the same construct to be introduced into the plant cell.

61. The method of claim 59, wherein the isolated fragment of claim 1 and the sequence to be transcribed are present on different constructs which are introduced into the plant cell, such that the promoter and coding sequence are placed in operable linkage in a plant cell following integration into the host cell genome.

62. The method of claim 59, wherein the sequence to be transcribed is endogenous to the plant cell and introduction of the isolated fragment of claim 1, and subsequent integration into the host cell genome sufficiently close to a target gene results in transcription of the endogenous sequence under the control of the promoter fragment of claim 1.

63. A method of altering a plant, the method comprising the introduction into the plant of an isolated fragment in accordance with claim 1.

64. A method according to claim 63, wherein performance of the method causes the formation of embryonic tissues in a mature plant.

65. A method according to claim 63, wherein performance of the method results in the accumulation of starch or another storage molecule in a mature plant.

66. A method of altering a plant in accordance with claim 65, wherein the isolated promoter fragment in accordance with claim 1 is introduced into a plant cell and placed in operable linkage with a nucleic acid coding sequence, and generating a plantlet and/or a plant from the transformed plant cell.

67. An altered plant cell produced by the method of claim 66.

68. An altered plant or plantlet, or the progeny thereof, produced by the method of claim 66, wherein the progeny of the plant of plantlet retain the introduced promoter fragment.


SEQUENCE LISTING (9 sequences)

SEQ ID NO: 1 - DNA, 23 bp, Artificial
  PCR Primer
rcargaymrnywbatgccrathg

SEQ ID NO: 2 - DNA, 23 bp, Artificial
  PCR Primer
cvsymtcdadyycvckrtavckd

SEQ ID NO: 3 - DNA, 30 bp, Artificial
  PCR Primer
gaattcccataacgcgttggtactctacgc

SEQ ID NO: 4 - DNA, 30 bp, Artificial
  PCR Primer
ctgcagcttggtggacaaacaagttaaggg

SEQ ID NO: 5 - DNA, 2814 bp, Arabidopsis thaliana
ttttaaaactaattttcaatttcattaattaagaaacaaagaatttagtgaaacctatattttattaaattttaataaaatatatgactaaaataacgtcacgtcaatctttctcagccgttcgataatcgaatactttattgactaaataaatagaaaatgttaaacaacatttaatttatataaacaaagagagcctcatatgtataaaaatcttcttcttatctttctttctttcttaatagtctttatttttacttaattactttggtaatttgtgaaaaaaaacaaccaatgagagaagagcagtttgactggccacatagccaatgagacaagccaatgggaaagagatatagacctcgtaagaaccgcctcctttgccatttgtatcatctctctataaaaccactcaaccatcaacctctctttgcatgcaacaaatcactcaaataattattttataaagaacaaaaaaaaaaaaagacggcagagaaacaatggaacgtggagctcccttctctcactatcagctaccaaaatccatctctggtaatctaagtggctatttttatacagtatatacttgcctccatgtatatttatatgctcatgaaaaattggagacatgctttatgaattttatgagactttgcaacaacgaactaaatgctttctttctagaaatttttaatttagatttgtgaaggttttgggaatggcccggagaagacgattttatatacatacatgcaagagtttgatatgtattgtttcatcatggctgagtcaaagttttatccaaatatttccatggtgtggtattagttaaacaaatctctcgtatgtgttcattgaatatacccgtgcatgtaccaggaatgtttttgagtatcgttttctttcttttatcaaaaaaaatttcgattctaaaaacgtttttttctttgttgtaacggttgagtttttttcttcgtttcaaaacgagattctcgtttgtctcttcccttgtctaaaaacatctacggttcatgtgattcaaaaacactaaaaaaatagaaactcaaatttttttagtacttaacatttaaactatatatatatatatatatatcttatactagtcccaagttttagtgtgaggtttttttattcaaaatctatcagtaatttttttggaaaaaaactaagtgaaattttctccaaattttccttttactattgattttttaattactggatgtcattaactccaatcttttgattctttcaacatttaccattgggaaccttcacatgaaataaatgtctactttattgagtcataccttcgtctacataaattaattgatgttcttctccaaattttgagtttttggtttttctaattaataatctaagcgaaagctttttggtatacatgtaaaacgtaacggcaagaatctgaacagtctactcaacggggtccataagtctagaatgtagaccccacaaacttactcttatcttattggtccgtaactaagaacgtgtcccttgattctcttgttttcttctaatccattcgtatcctacaaatttaatcatcatttctacttcaactaatcttttttatttcctaaagatttcaatttctctctgtattttctatgaacagaattgaacttggaccagcacagcaacaacccaaccccaatgaccagctcagtcgtagtagccggcgccggtgacaagaacaatggtatcgtggtccagcagcaaccaccatgtgtggctcgtgagcaagaccaatacatgccaatcgcaaacgtcataagaatcatgcgtaaaaccttaccgtctcacgccaaaatctctgacgacgccaaagaaacgattcaagaatgtgtctccgagtacatcagcttcgtgaccggtgaagccaacgagcgttgccaacgtgagcaacgtaagaccataactgctgaagatatcctttgggctatgagcaagcttgggttcgataactacgtggaccccctcaccgtgttcattaaccggtaccgtgagatagagaccgatcgtggttctgcacttagaggtgagccaccgtcgttgagacaaacctatggaggaaatggtattgggttcacggcccatctcatggcctacctcctccgggtccttatggttatggtatgttggaccaatccatggttatgggaggtggtcggtactaccaaaacgggtcgtcgggtcaagatgaatccagtgttggtggtggctcttcgtcttccattaacggaatgccggcttttgaccattatggtcagtataagtgaagatggaattattcttcatttttatatctgttcaaaacatgtgtttggatagatattttatttttatgtcttatcaataacatttctatataatgttgcttctttaaggaaaagtgttgtatttcaatactttatgagaaactgatttatatatgcaaatgatttaacccaaactgttttgtggattaaactctatgcaacattatatatttacatgatctaaaggttttgtaattcaaaagctgtcatagttagatgataactaaacattgtaggaaccaagtttaattgacttttttgagttttacacaactaagccaaaggattataaaatctaaattcgttgagttgtcaaacttctgaagatttatatcctctttgagtttgctttcttttgggtgcttgagtttcattagggtgatcctgactcgttgctctctggtttctccatctctgtcttttccaaggattcataacgttggtcgctctctgt

SEQ ID NO: 6 - DNA, 6680 bp, Arabidopsis thaliana
gagttttaacagatttcttaacaaaaaaaactcagtggagaaacgaaaaacacatttctccggcgattcaccaccggatttttcaaatgggtttgaataaagctagaaactttatcaagaatttggttactcagaagatatcacacaagtatttctaaatctgagaagaagaaaccttgtgagatttcgtacagacgctacaagaacatcaaaagaagaaaagtgatgaacagagagacacaagagagacgatgattaatttttaatttataaaataaaagtattgggaagcggacacaaaaagcccaccagcccgaaaaagaaacccacaatattattaaaaatttgaaaacgacaccgtagtgtaaacataaaattaataatacatacgtactcgagattgtttcgaatttgtctttgtataatttaaagcgatatgatatagtatgggctacttcatacgttaatagtagaagaccaaatgattagtttatggtctacctttgtttacttattttattgatagtagagaaatgattatgattgagaggattatcgagaaatcttgattaggaagcgagtttccaggtgattgtctttgatgacttacttaattaattacttttatgrttctaagaattttgttctcttctgtgattagatgatcgttgtcttggtgggttttttttatatcttttcgagtattgattctcttcctacttgcgatgatttattaatttaaaggatacttatattcccatacggatagtgatagatacagaagatggttgatgcaagtagtatatgaaattctaaagttgcgttatttttttaacgtttttatgttgatattgatagtgccaaaggtttacattttaaagttgagtttttgtccgccatttgtaaattaggtctaatattttatttgataaataatcgagattttttatagtttggtttcgcataaatagacatacatcatcctataggctataggttcgtgtactttgatttgaagtgctagtagtaggacatgattgtcattcgaataaccacaaacatcataaaaactatattgcgagttgcgaccgtgcacgtaaacttcataattcaaacgtgtaacattttaacaaaaaaaaatgctacaaaaatacctagtacaccacatattaagtatgtatatttctaaaattgtattatacgtacgagtggtcagtggttagttaccacggctgtggtggttagagtatctaacacatttgatcaccatttaattaaaatctttataaaaatatattacaaccgcattaggaaaaattaaaaatatacttcaagggaacgtgggcgtacgtaatctgagtttaaactttttgtataaaagaagcaattgaatcactcaaaatgttcttaaaaccttaattcaagtttgatactccttgtttttatatcttcataaactgaccatttaacatttgttttcttgcgtttgttctacgtgttggagctattcgacacttatttatgttatttcgtcattactacaccgtttattttatttcggtactcgaagccgacgttaatgctgggggtggatccccagttaaaacctaaaagtactcaataacttttgtccacattaatgaaaaaatcacacgatacgaataaatataaatattaggagtataaaatatggttgaatgaatgtatcttttcccattcatacaccataacgcgttggtactctacgccatactgagatactttcccattccatattcaaggcatctcattactcttcgctgtatgcctgtatacttgaaatctgaaagctctagtgtttcttttttgacgctttcaattctaaaaacccacgaggacacatctttttcgattcctagccatcattttattgtatatatgatttttatattgtcatagaatacacattttgatttgcaaaaaaaaaaaacattcttagcgaaataccgacttaattgtccacaaatatgtaagataagatctaattatattgatgtattcaatcacacgtattggcatacttataaactgtggtacttcgaatctgatgtaaaacatctttgtttttttttggtcaactactaagagaatctctaaaattagtcttacacaaaactttattttattgagtactgatttcgaaagaaacaagtgtcttcggttacttgtttttgacgaaaataaaatattgccttaggcaaattaaaaacattaaacacgaatgttacgtgtttaattggctaagttaacgcaaaaaaatacatatacatatatttgatatattcagcataatttgttttatttatcgtttttagaattcgtggccattagacccataactatatgacgatgttaaagagaaaataaatcataaataaaataagagtccttatcaataaacctaattggctaatttcaacctcaaagagtagtaggaacaggtaaggtgaagccaaacagctccttttacagttggaccactagagctgatctggcatacaaagtatgcttattgggctgtcacggcccatccgcaaaatgtcgttggttacgaagcatccacgacatagacggtgccacatgtagaaaagtgtttcggcgatcaagattgtgtccacatcattagacgtctgaactgtccacgtgtctatcaaagctggcgtcaaacattacgttttcgtcgtttgcgcctcctagttcacacgtgcaacgaacgcgtgcgacgtatcaaaattgttaattttagccatgtataaagaatatctacaaaattaacctcaggaatatttttgttttttcaattgaggccataatatacgttccgattgagagatttttcatcatatcactaatatcaaaagattatgatgttagtaaaacgtagaaaatttacacaaaataaatttcacaaaacttaatggggaaattgaaacaaagaaaagactggtgagtgataagcgatgatggccggtgaatcaggtagccgtcctacaacgtggttgattttgagcaaactcctatttactcttcacactattgaaatcccaaaatgtcgtcacaccataataatgtgaattttgttatggaatttgagggaaacagtagatatatgtttcaaccagtgaaagttaccctgctttggacatatctacgagagtagaaagtagaaacattcactaaacgtgacaactttataaattttctttttgtaacttttctttagatttatttacgagaagagaaatataaacgtcatgctaataaaaaatgcattattttctaccatctagctagaatattgatcaagtcttcacgttttttgtttatctcttctctcataggcatgtccacaaaagggtaagttttactggttcaaaatattgcatgagtactactaagctcgtatagtttgatcttaatatcattgcgatgagggttgttagtttggaagaaataaggatttatgcaaatggtaatcattatgtctgctatttaagaattaaattatgatgcttgttgcgtgaacatattaaatttgcgaaaaaaaagcaaggatacacgagagaagctcagatattcacgtaacgatgtttcatctcttctcattgaggaaacatatggccatggatatagctattaagcctacgggattgtcatttcaacgccgaatctaccaaactgttccatctcttattatatatagtttggttatttaagtaattagatgcatcataatctttttttctgccagttgtaatgcagataaaaatatattggttgttctaaggattgttcaaacgtgcatgcgtacaagttattatttatatactttcatctacatgcgatgcgttatttataatgataaaactaagatttttagttaaatttaataaagagcttacgagctacaattaattagaaatggttgctcagaaatcagaatactatatatgaaaaaagaagttggtatacttgaaaaaagaaaaaactacttgaaaagatggtaaaagatatagaacgagtatatatcttactcaagcacgatagaagtttgtatcaaaacattgcgttccaaaccaatgtttgaagatggtcaaaggtgctactcatgatgtatgcgaagaagcttacaaaaaattctgcaatgagagataactttatgggctgcttgttcaatatattgaaaatcatggtagacaacaccaaactctcctttaccagaagtcatatttccttaacctcagaataagtaaatcttctagtttattatttgaaagctgagcgtataattgcaatgaaacttttaccaattcaccgcctcctaactgagttgttgtattatcctatctctttagctatcctttccttgctcttgctccacctgcatgtggcctctttatttataatctctctagattctgctaaagatgtatgttcaaaatggtttatctttaagggaagcaaagtgaatggaaacatttaaagaaaaaaaaaacttttagcagagttccatgagatttcatactgatgataactaaaataatcttatatgcgtaagattattttagttctaaacttcattttgaaatgagaggtcattggccaggaaagattcaatattggttctttgttaattctcgttggtttgtttttagtatgggctagatccaaaacaggtcatggactgggccgtaaactctatccaaaattcttcatgtttttccatctttcaaaaatctttatccaccattccattactagggtgttggttttattttatttgttgattaattatgtattagaaaatgtaaagcaatattcaattgtaacatgcatcatctaacaccaatatcttgtactaaccttttgtaattttcctataaacactttaaaaggctaatttaaataaaaattacaataaacgtgataactcattttcgtaacgcatatttattcaaatataccaaaatttacattttaagtaagagaatctttttaaaactaattttcaatttcattaattaagaaacaaagaatttagtgaaacctatattttattaaattttagtaaaatatatgactaaaataacgtcacgtgaatttttctcagccgttcgataatcgaatactttattgactaaataaatagaaaatgttaaacaacatttaatttatataaacaaagagagcctcatatgtataaaaatcttcttcttatctttctttctttcttaatagtctttatttttacttaattactttggtaatttgtgaaaaaaacaaccaatgagagaagagcagtttgactggccacatagccaatgagacaagccaatgggaaagagatatagagacctcgtaagaaccgcctcctttgccatttgtatcatctctctataaaaccactcaaccatcaacctctctttgcatgcaacaaatcactcaaataattattttataaagaacaaaaaaaaaaagacggcagagaaacaatggaacgtggagctcccttctctcactatcagctaccaaaatccatctctggtaatctaagtggctatttttatacagtatatacttgcctccatgtatatttatatgctcatgaaaaattggagacatgctttatgaattttatgagactttgcaacaacgaactaaatgctttctttctagaaatttttaatttagatttgtgaaggttttgggaatggcccggagaagacgattttatatacatacatgcaagagtttgatatgtattgtttcatcatggctgactcaaagttttatccaaatatttccatggtgtggtattagttaaacaaatctctcgtatgtgttcattgaatatacccgtgcatgtaccaggaatgtttttgagtatcgttttctttcttttatcaaaaaaaatttcgattctaaaaacgtttttttctttgttgtaacggttgagtttttttcttcgtttcaagacgagattctcgtttgtctcttcctttgtctaaaaacatctacggttcatgtgattcaaaaacactaaaaaaatagaaactcaaatttttttagtacttaacatttaaactatatatatatatcttatactagtcccaagttttagtgtgaggtttttttattcaaaatctatcagtaatttttttggaaaaaaactaagtgaaattttctccaaattttccttttactattgattttttaattactggatgtcattaactccaatcttttgattctttcaacatttaccattgggaaccttcacatgaaataaatgtctactttattgagtcataccttcgtctacataaattaattgatgttcttctccaaattttgagtttttggtttttctaattaataatctaagcgaaagctttttggtatacatgtaaaacgtaacggcaagaatctgaacagtctactcaacggggtccataagtctagaatgtagaccccacaaacttactcttatcttattggtccgtaactaagaacgtgtcccttgattctcttgttttcttctaatccattcgtatcctacaaatttaatcatcatttctacttcaactaatcttttttatttcctaaagatttcaatttctctctgtattttctatgaacag

SEQ ID NO: 7 - protein, 238 aa, Arabidopsis thaliana
MERGAPFSHYQLPKSISELNLDQHSNNPTPMTSSVVVAGAGDKNNGIVVQQQPPCVAREQDQYMPIANVIRIMRKTLPSHAKISDDAKETIQECVSEYISFVTGEANERCQREQRKTITAEDILWAMSKLGFDNYVDPLTVFINRYREIETDRGSALRGEPPSLRQTYGGNGIGFHGPSHGLPPPGPYGYGMLDQSMVMGGGRYYQNGSSGQDESSVGGGSSSSINGMPAFDHYGQYK

SEQ ID NO: 8 - protein, 208 aa, Arabidopsis thaliana
MTSSVVVAGAGDKNNGIVVQQQPPCVAREQDQYMPIANVIRIMRKTLPSHAKISDDAKETIQECVSEYISFVTGEANERCQREQRKTITAEDILWAMSKLGFDNYVDPLTVFINRYREIETDRGSALRGEPPSLRQTYGGNGIGFHGPSHGLPPPGPYGYGMLDQSMVMGGGRYYQNGSSGQDESSVGGGSSSSINGMPAFDHYGQYK

SEQ ID NO: 9 - DNA, 3529 bp, Arabidopsis thaliana
gaattcccataacgcgttggtactctacgccatactgagatactttcccattccatattcaaggcatctcatttctgatttcattaattaagaaacaaagaatttagtgaaacctatattttattaaattttagtaaaatatatgactaaaataacgtcacgtgaatttttctcagccgttcgataatcgaatactttattgactaaataaatagaaaatgttaaacaacatttaatttatataaacaaagagagcctcatatgtataaaaatcttcttcttatctttctttctttcttaatagtctttatttttacttaattactttggtaatttgtgaaaaaaacaaccaatgagagaagagcagtttgactggccacatagccaatgagacaagccaatgggaaagagatatagagacctcgtaagaaccgcctcctttgccatttgtatcatctctctataaaaccactcaaccatcaacctctctttgcatgcaacaaatcactcaaataattattttataaagaacaaaaaaaaaaaagacggcagagaaacaatggaacgtggagctcccttctctcactatcagctaccaaaatccatctctggtaatctaagtggctatttttatacagtatatacttgcctccatgtatatttatatgctcatgaaaaattggagacatgctttatgaattttatgagactttgcaacaacgaactaaatgctttctttctagaaatttttaatttagatttgtgaaggttttgggaatggcccggagaagacgatttcatatacatacatgcaagagtttgatatgtattgtttcatcatggctgactcaaagttttatccaaatatttccatggtgtggtattagttaaacaaatctctcgtatgtgttcattgaatatacccgtgcatgtaccaggaatgtttttgagtatcgttttctttcttttatcaaaaaaaatttcgattctaaaaacgtttttttctttgttgtaacggttgagtttttttcttcgtttcaaaacgagattctcgtttgtctcttcctttgtctaaaaacatctacggttcatgtgattcaaaaacactaaaaaaatagaaactcaaatttttttagtacttaacatttaaactatatatatatatcttatactagtcccaagttttagtgtgaggtttttttattcaaaatctatcagtaatttttttggaaaaaaactaagtgaaattttctccaaattttccttttactattgattttttaattactggatgtcattaactccaatcttttgattctttcaacatttaccattgggaaccttcacatgaaataaatgtctactttattgagtcataccttcgtctacataaattaattgatgttcttctccaaattttgagtttttggtttttctaattaataatctaagcgaaagctttttggtatacatgtaaaacgtaacggcaagaatctgaacagtctactcaacggggtccataagtctagaatgtagaccccacaaacttactcttatcttattggtccgtaactaagaacgtgtcccttgattctcttgttttcttctaatccattcgtatcctacaaatttaatcatcatttctacttcaactaatcttttttatttcctaaagatttcaatttctctctgtattttctatgaacagaattgaacttggaccagcacagcaacaacccaaccccaatgaccagctcagtcgtagtagccggcgccggtgacaagaacaatggtatcgtggtccagcagcaaccaccatgtgtggctcgtgagcaagaccaatacatgccaatcgcaaacgtcattagaatcatgcgtaaaaccttaccgtctcacgccaaaatctctgacgacgccaaagaaacgattcaagaatgtgtctccgagtacatcagcttcgtgaccggtgaagccaacgagcgttgccaacgtgagcaacgtaagaccataactgctgaagatatcctttgggctatgagcaagcttgggttcgataactacgtggaccccctcaccgtgttcattaaccggtaccgtgagatagagaccgatcgtggttctgcacttagaggtgagccaccgtcgttgagacaaacctatggaggaaatggtattgggtttcacggcccatctcatggcctacctcctccgggtccttatggttatggtatgttggaccaatccatggttatgggaggtggtcggtactaccaaaacgggtcgtcgggtcaagatgaatccagtgttggtggtggctcttcgtcttccattaacggaatgccggcttttgaccattatggtcagtataagtgaagatggaattattcttcatttttatatctgttcaaaacatgtgtttggatagatattttttttttatgtcttatcaataacatttctatataatgttgcttctttaaggaaaagtgttgtatttcaatactttatgagaaactgatttatatatgcaaatgatttaacccaaactgttttgtggattaaactctatgcaacattatatatttacatgatctaaaggttttgtaattcaaaagctgtcatagttagatgataactaaacattgtaggaaccaagtttaatttacttttttgagttttacacaactaagccaaaggattataaaatctaaattcgttgagttgtcaaacttctgaagatttatatcctctttgagtttgctttcttttgggtgcttgagtttcattagggtgatcctgactcgttgctctctggtttctccatctctgtcttttccaatgattcataacgttggtcgctctctgtttctgcctacacttcttcaaggatcattactaagactaagagttaaagaccttagccatggttttctgtaactggttcaagttcatctccggttattgggtggttatctttcggttagattgaaacccatatgcttgctctgtttcttctagttccaagtttaatttccggttattgtttggctttttaaagttcttaaaggtctattctatgtaaagactcttttgaaattttatggctgaatgaaaaaagtccctttaagtctttaatttgtgttgatgtttaggatacttcgacaatcttaagaaaatcaagggctaaaaatgaaagcaagttaccaaattactcaaaatctaaaaaaagtgacaaatattttagcccaatggattaaactaaaatgtagtttcttttatcattttaggctatgttttcttttaagaaaactttggtagttaactctgtttaaaagaaaaaatagagatgcttaaattaaatttagtatctaaaactttaggataaacacattaagctaaagaaattaaattaacaggcgtaatgcaagcttgttatgcggtattgaaaacagtacctctaaaagtagcccaatattgaaaaacttaagcttctttgatccccttaacttgtttgtccaccaagctgcag